(19) United States
(12) Patent Application Publication
(10) Pub. No.: US 2006/0263438 A1
(43) Pub. Date: Nov. 23, 2006

(54) Gelled oil particles for targeting sebaceous glands and/or hair follicles

(75) Inventors: Bruno Biatry, Vincennes (FR); Jean-Thierry Simonnet, Cachan (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/434,828

(22) Filed: May 17, 2006

Related U.S. Application Data
(60) Provisional application No. 60/688,323, filed on Jun. 8, 2005.

(30) Foreign Application Priority Data
May 17, 2005 (FR) 05 51275

(51) Int. Cl. A61K 9/50; A61K 9/16

(52) U.S. Cl. 424/490

(57) ABSTRACT

The present invention relates to calibrated and spherical oily particles comprising at least one oily phase structured with at least one gelling polymer, characterized in that they have a mean size of less than or equal to 15 μm, in that the said structured oily phase has a melting point of greater than or equal to 40° C. and in that their circularity index is between 0.9 and 1.

### CROSS-REFERENCE TO RELATED APPLICATIONS

This non provisional application claims the benefit of French Application No. 05 51275 filed on May 17, 2005 and U.S. Provisional Application No. 60/688,323 filed on Jun. 8, 2005, the disclosures of which are incorporated herein by reference in their entireties.

### BACKGROUND

The present invention relates to calibrated and spherical oily particles, especially suitable for targeting hair follicles and/or sebaceous glands.

The present invention also relates to a cosmetic or dermatological composition comprising these particles, and also to a process for manufacturing these particles.

Hair follicles and sebaceous glands are generally combined in a “pilosebaceous” unit, although certain regions of the skin may comprise sebaceous glands not associated with hairs. With the exception of the palms of the hands and the soles of the feet and of the toes, sebaceous glands are present throughout the skin, including the lips. Their density varies as a function of the region under consideration.

This pilosebaceous unit, or follicle, is formed from a tubular invagination of the epidermis, which is buried in the dermis. The sebaceous glands secrete a liquid product, sebum, which can have an influence on the greasy or dry nature of the skin. The pilosebaceous unit can become subject to an inflammatory process, which especially impairs the appearance of the skin, and the causes of which may be varied. For example, it may be brought about by the presence of microorganisms.

As a result of its morphology, this unit can constitute a potential passageway for agents intended to act either on deep skin tissues, for example for agents of the type for combating deep wrinkles or of the slimming type, or on the surface skin tissues, for which it acts as a reservoir of substance, or, where appropriate, to allow the penetration of active substances into the blood stream and to allow their diffusion throughout the body.

Various methods specifically aimed at exploiting this passageway for targeting substances that are active on the hair follicles and/or the sebaceous glands have already been proposed. The sebaceous glands must thus be used as a reservoir of these active substances, which are then released to reach their site of action, for instance the skin or the hair.

Thus, polymer particles having a network of pores capable of containing an active molecule intended to be released from the particle have been proposed (see U.S. Pat. No. 4,690,825). These polymer particles are obtained via polymer polymerization of styrene and divinylbenzene type, and have a diameter ranging from 10 to 100 microns.

Capsules comprising microparticles impregnated with an active substance dispersed in an oily phase of silicone type, which is optionally thickened or gelled, have also been described (see WO 99/53904). These microparticles are obtained by polymerizing a mixture of unsaturated monoethylenic monomers or unsaturated polyethylenic monomers in the presence of a pore-forming agent so as to obtain a network of pores. These capsules have a mean diameter from about 1 μm to about 500 μm.

However, the microparticles described in the documents mentioned above have the drawback either of requiring a solvent in order to be filled with active substances, or of requiring the introduction of the active substance during the polymerization step, thus entailing a risk of degradation of the active substance. Moreover, the use of these porous particles may impose formulation constraints intended to limit the leakage of the active substances into the rest of the composition.

Particles of oily nature have been proposed as an alternative.

Thus, the use of particles with a diameter from about 50 μm to 10 mm and comprising an oil structured with a gelling agent to formulate a composition for cleansing the skin and the hair has been proposed (see U.S. Pat. No. 6,737,394).

WO 02/092043 discloses a skincare composition comprising an aqueous phase in which is dispersed an oily phase structured with a gelling agent. The oily phase, whose viscosity does not exceed 500 Pa·s, is dispersed therein in the form of particles ranging from 1 to 500 μm in size.

EP 0 375 520 discloses a cosmetic composition for topical application comprising particles of fatty substance, comprising an active product, and having a diameter of between 3 and 10 μm.

However, all of the particles described previously are obtained by means of processes that do not allow the production of particles that are, on the one hand, calibrated in a predetermined size range, and, moreover, compatible for optimum targeting of hair follicles and/or sebaceous glands.

Now, the calibration of the particles is specifically advantageous insofar as it ensures that they have uniform size and shape distributions and increased stability and performance qualities, especially in terms of vehicle for active substances.

For the purposes of the present invention, the term “active substances” is intended to denote molecules or substances capable of exerting an effect, after application to a part of the human or animal body.

### SUMMARY

There is consequently a need to optimize the targeting and the penetration of active substances into the sebaceous glands and/or the hair follicles.

There is also a need to improve the stability of the loading of the active substance into particles capable of penetrating the sebaceous glands and/or the hair follicles.

There is also a need to obtain structured oily particles for targeting the sebaceous glands and/or the hair follicles, which are stable and which do not exude over time.

There is also a need to obtain oily particles capable of penetrating the sebaceous glands and/or the hair follicles, which are homogeneous in their structure and their size distribution.

There is also a need to allow a release of active substances from the sebaceous glands and/or the hair follicles according to an optimum time cycle.

There is also a need to obtain oily particles for storing and gradually releasing active substances from the sebaceous glands and/or the hair follicles, so as to promote prolonged contact of these substances with their site of action, and thus to improve their efficacy.

There is also a need to obtain oily particles whose integrity is maintained during application to a support.

The object of the present invention is, precisely, to satisfy all or some of the needs by overcoming the drawbacks mentioned above.

The inventors have observed that it is possible to obtain, from an oily phase structured with at least one gelling polymer, calibrated and spherical oily particles that are conformed so as to efficiently target the sebaceous glands and/or the hair follicles, and especially to promote the penetration of active substances therein, and thus to increase the efficacy of these active substances.

According to one of its first aspects, one subject of the present invention is thus calibrated and spherical oily particles comprising at least one oily phase structured with at least one gelling polymer, the said particles having a mean size of less than or equal to 15 μm and the said structured oily phase having a melting point of greater than or equal to 40° C., and their circularity index being between 0.9 and 1.

A subject of the present invention is also calibrated and spherical oily particles comprising at least one oily phase structured with at least one gelling polymer and having a mean size of less than or equal to 15 μm, the gelling polymer being chosen from a semi-crystalline polymer, a polyamide, a silicone polyamide, a polysaccharide monoalkyl ester or polyalkyl ester with the exception of fatty acid esters of dextrin, a diblock and/or triblock and/or multiblock and/or radial-block copolymer, and mixtures thereof, and their circularity index being between 0.9 and 1.

According to another of its aspects, a subject of the present invention is also calibrated and spherical oily particles obtained from an oily phase structured with at least one gelling polymer, the gelling polymer being of a nature and/or in an amount sufficient to give the said oily phase a viscosity of greater than or equal to 750 Pa·s at a shear of 1 s⁻¹, at 25° C., and their circularity index is between 0.9 and 1.

For the purposes of the present invention, the term “sufficient content” is intended to denote the minimum content required to observe the expected effect, i.e., in the case of the gelling polymer, the production of a structured oily phase having the viscosity required to obtain particles in accordance with the present invention.

The calibrated and spherical particles according to the invention may comprise at least one active substance, which may be, by means of the conformation of the particles, advantageously targeted towards the hair follicles and/or the sebaceous glands.

According to yet another of its aspects, a subject of the present invention is a dispersion in aqueous and/or water-soluble phase comprising particles in accordance with the present invention.

According to yet another of its aspects, a subject of the present invention is also a process for manufacturing a dispersion in accordance with the invention.

According to yet another of its aspects, a subject of the present invention is a cosmetic or dermatological composition comprising at least some particles and/or at least a dispersion in accordance with the invention.

According to yet another of its aspects, a subject of the present invention is also the use of particles and/or of at least one dispersion in accordance with the invention in a cosmetic composition for treating greasy skin.

According to another of its aspects, a subject of the present invention is also the use of particles and/or of at least one dispersion in accordance with the invention for the preparation of a dermatological composition for treating acne.

According to yet another of its aspects, a subject of the present invention is also a non-therapeutic process for making up and/or caring for the skin, comprising at least one step of applying at least one composition in accordance with the invention thereto.

It is understood that the compositions in accordance with the invention are advantageously intended to be applied to any part of the skin of the human or animal body, comprising at least one sebaceous gland and/or at least one hair follicle, i.e. any part of the skin, including the lips and the scalp.

In the context of the present invention, the terms “gelled” and “thickened” may be considered as being synonymous with the term “structured” when it is a matter of qualifying the oily particles.

All cited references are incorporated herein by reference in their entireties.

### DETAILED DESCRIPTION OF EMBODIMENTS

Calibrated and Spherical Particles

The calibrated oily particles comprising at least one structured oil or oily phase in accordance with the invention have a mean size of less than or equal to 15 μm, in particular less than or equal to 12 μm and more particularly less than or equal to 11 μm. Advantageously, the mean size of the particles may range from 100 m to 15 μm, from 100 nm to 12 μm or even from 150 nm to 11 μm.

For the purposes of the present invention, the term “calibrated” refers to particles having a homogeneous granulometric distribution.

The granulometric distribution qualifies the distribution of the size of the calibrated particles about a mean size. The granulometric distribution may be characterized by a polydispersity index or a coefficient of uniformity. The lower the index or the coefficient, the more uniformly the particle sizes are distributed around a mean size.

Thus, for the submicron calibrated oily particles, in accordance with the invention, i.e. with a mean size of less than 1 micrometer, the granulometric distribution may be characterized by a polydispersity index, noted as PI (dimensionless value characterizing the extent of the granulometric distribution). This index is then advantageously less than or equal to 0.35 and preferably greater than or equal to 0.01.

The size of the submicron calibrated oily particles may be determined, for example, with a laser granulometer functioning on the principle of quasi-elastic light scattering, for instance the BI90Plus® machine from Brookhaven Instrument.

For the calibrated oily particles with a mean size of greater than one micrometer, the granulometric distribution may be characterized by a coefficient of uniformity measured using a laser-scattering granulometer, for instance the Master Sizer 2000® machine from Malvern. The calibrated oily particles in accordance with the invention that are greater than one micrometer in size may have a coefficient of uniformity advantageously of less than or equal to 0.45 and preferably greater than or equal to 0.1.

The size of the particles and the homogeneity of the granulometric distribution about a mean size are generally determined by the nature of the process used to obtain them. The processes for obtaining the calibrated particles in accordance with the invention are described hereinbelow.

The calibrated particles have a uniform and substantially spherical shape.

The term “substantially spherical” means that the particles are of substantially isotropic shape, i.e. they have a relatively regular morphology.

In the context of the present invention, a parameter relative to the shape factor of the particles is thus defined, for instance the circularity index C, which is defined as the ratio of the total surface area A of the particle to the surface area of the disc having the same perimeter P:
C=4□A/P

with C between 0.9 and 1.

This measurement may advantageously be performed using a Sysmex FPIA 2100 machine, which is an image analysis granulometer.

Moreover, besides the homogeneity of shape and of size distribution, the calibrated and spherical particles in accordance with the invention are advantageously homogeneous as regards their structure.

Thus, the oily gel constituting the particle and obtained by structuring at least one oil or oily phase with at least one gelling polymer, advantageously has a uniform structure.

Advantageously, when an active substance is incorporated into the particles in accordance with the present invention, the distribution of this active substance throughout the particle is also homogeneous.

Oil

The calibrated and spherical oily particles in accordance with the present invention comprise at least one oil or oily phase, especially containing at least one oil that is liquid at room temperature (20-25° C.) and at atmospheric pressure.

For the purposes of the present invention, the term “oily phase” is intended to denote a phase comprising at least one oil. This oil is advantageously a non-volatile oil. For the purposes of the present invention, the term “non-volatile oil” means an oil having a vapour pressure of less than 0.13 Pa.

The oily phase that is suitable for use in particles in accordance with the present invention may comprise, for example, at least one oil chosen from a plant oil, an animal oil, a synthetic oil and a mineral oil, and mixtures thereof.

The oily phase may also comprise at least one volatile oil, which may require particular processing conditions, especially under pressure.

For the purposes of the present invention, the term “volatile oil” means an oil (or non-aqueous medium) that is capable of evaporating on contact with the skin (for example at about 33° C.) in less than one hour, at room temperature and atmospheric pressure. The volatile oil is a volatile cosmetic oil, which is liquid at room temperature, especially having a non-zero vapour pressure, at room temperature and atmospheric pressure, in particular having a vapour pressure ranging from 0.13 Pa to 40 000 Pa (10⁻³ to 300 mmHg), preferably ranging from 1.3 Pa to 13 000 Pa (0.01 to 100 mmHg) and preferentially ranging from 1.3 Pa to 1300 Pa (0.01 to 10 mmHg).

The oils that are suitable for use in the invention may be of natural, plant or mineral origin, or of synthetic origin. They may be of hydrocarbon-based type, for instance triglycerides, esters, alkanes or polyolefins, of silicone type or of fluoro type, and may be modified or non-modified.

For the purposes of the present invention, the term “fluoro oil” means an oil comprising at least one fluorine atom.

For the purposes of the present invention, the term “silicone oil” means an oil comprising at least one silicon atom, and especially at least one Si—O group.

The term “hydrocarbon-based oil” is intended to denote an oil mainly containing hydrogen and carbon atoms and possibly oxygen, nitrogen, sulfur and/or phosphorus atoms.

According to one embodiment, they may be used alone or as a mixture, with each other or with other compounds as defined, for example, hereinbelow.

Advantageously, the oils used for the implementation of the invention are compatible with the gelling polymer used to structure the oily phase.

The non-volatile oils may especially be chosen from non-volatile hydrocarbon-based oils and, where appropriate, fluoro oils and/or silicone oils.

Non-volatile hydrocarbon-based oils that may especially be mentioned include:
hydrocarbon-based oils of animal origin, such as squalane;
hydrocarbon-based oils of plant origin such as phytostearyl esters, such as phytostearyl oleate, phytostearyl isostearate and lauroyl/octyldodecyl/phytostearyl glutamate (Ajinomoto, Eldew PS203); triglycerides consisting of fatty acid esters of glycerol, the fatty acids of which may have chain lengths ranging from C₄ to C₂₄, these chains possibly being linear or branched, and saturated or unsaturated; these oils are especially heptanoic or octanoic triglycerides, wheatgerm oil, sunflower oil, grapeseed oil, sesame oil, corn oil, apricot oil, castor oil, shea oil, avocado oil, olive oil, soybean oil, sweet almond oil, palm oil, rapeseed oil, cotton seed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil or musk rose oil; shea butter; or alternatively caprylic/capric acid triglycerides, for instance those sold by the company Stearineries Dubois or those sold under the names Miglyol 810®, 812® and 818® by the company Dynamit Nobel, and mixtures thereof;
linear or branched hydrocarbons of mineral or synthetic origin such as petroleum jelly, polydecenes, hydrogenated polyisobutene such as Parleam, and mixtures thereof;
synthetic ethers containing from 10 to 40 carbon atoms;
synthetic esters, for instance oils of formula R₁ COOR₂ in which R₁ represents a linear or branched fatty acid residue containing from 1 to 40 carbon atoms and R₂ represents a hydrocarbon-based chain that is especially branched, containing from 1 to 40 carbon atoms provided that R₁+R₂≧10;
and mixtures thereof.

The esters may be chosen especially from fatty acid esters, for instance:
cetostearyl octanoate, isopropyl alcohol esters, such as isopropyl myristate, isopropyl palmitate, isopropyl lauroylsarcosinate, ethyl palmitate, 2-ethylhexyl palmitate, isopropyl stearate, isopropyl isostearate, isostearyl isostearate, octyl stearate, hydroxylated esters, for instance isostearyl lactate, octyl hydroxystearate, diisopropyl adipate, heptanoates and especially isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate, polyethylene glycol diheptanoate, propylene glycol 2-diethylhexanoate and mixtures thereof, hexyllaurate, neopentanoic acid esters, for instance isodecyl neopentanoate, isotridecyl neopentanoate, isostearyl neopentanoate and octyldodecyl neopentanoate, isononanoic acid esters, for instance isononyl isononanoate, isotridecyl isononanoate and octyl isononanoate, hydroxylated esters, for instance isostearyl lactate or diisostearyl malate, alkylbenzoate and C₁₂ to C₁₅ alcohol benzoates, and mixtures thereof;
polyol esters and pentaerythritol esters, for instance dipentaerythrityl tetrahydroxystearate/tetraisostearate;
esters of diol dimers and diacid dimers such as Lusplan DD-DA5® and DD-DA7®, and mixtures thereof, sold by the company Nippon Fine Chemical and described in patent application FR0 302 809 filed on 6 Mar. 2003, the content of which is incorporated into the present patent application by reference;
fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol;
higher fatty acids that are liquid such as oleic acid, linoleic acid or linolenic acid, and mixtures thereof; and
dialkyl carbonates, the two alkyl chains possibly being identical or different, such as dicaprylyl carbonate sold under the name Cetiol CC® by Cognis; and
mixtures thereof

The non-volatile silicone oils that may be used in the composition according to the invention may be non-volatile polydimethylsiloxanes (PDMS), such as simethicone, polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenylmethyl diphenyltrisiloxanes and 2-phenylethyl trimethylsiloxysilicates, and dimethicones or phenyl trimethicones with a viscosity of less than or equal to 100 cSt, and mixtures thereof.

The volatile hydrocarbon-based oils optionally present may especially be chosen from hydrocarbon-based oils containing from 8 to 16 carbon atoms, and especially branched C₈-C₁₆ alkanes (also known as isoparaffins), for instance isododecane (also known as 2,2,4,4,6-pentamethylheptane), isodecane, isohexadecane and, for example, the oils sold under the trade names Isopar® or Permethyl®.

As volatile oils optionally present, it is also possible to use volatile silicones, for instance volatile linear or cyclic silicone oils, especially those with a viscosity ≦8 centistokes (8×10⁻⁶ m²/s), and especially containing from 2 to 10 silicon atoms and in particular from 2 to 7 silicon atoms, these silicone oils optionally comprising alkyl or alkoxy groups containing from 1 to 10 carbon atoms. As a volatile silicone oil that may be used in the invention, mention may be made especially of dimethicones with a viscosity of 5 and 6 cSt, such as octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Volatile fluoro oils such as nonafluoromethoxybutane or perfluoromethyl-cyclopentane, and mixtures thereof, may also be used.

Advantageously, the oily phase present in the particles in accordance with the present invention may comprise at least one oil chosen especially from squalane, isononyl isononanoate, isopropyl lauroylsarcosinate and octyldodecanol, and mixtures thereof.

The content of oily phase in the particles in accordance with the invention may range from 10% to 99% by weight, in particular from at least 20% to 99% by weight and more particularly from 30% to 99% by weight relative to the total weight of the calibrated particle according to the invention.

According to one embodiment, the oily phase, structured with at least one gelling polymer forming calibrated oily particles in accordance with the invention, has a melting point of greater than or equal to 40° C. and preferably less than or equal to 95° C.

According to one preferred embodiment, the same oily phase has a melting point that may range from 50 to 90° C.

The structured oily phase from which the calibrated oily particles in accordance with the invention are derived advantageously has a viscosity of greater than 750 Pa·s at a shear of 1 s⁻¹ at 25° C., and preferably less than or equal to 1×10⁶ Pa·s.

The measurement may be performed using an RFS3 Rheometrics rheometer.

The measurements are then taken at 25° C., the temperature being regulated by the Peltier effect.

The geometry is a cone/plate geometry, with a cone 25 mm in diameter and an angle of 2°.

A rate gradient of 1 s⁻¹ is imposed, for a certain equilibrium time, for example 5 minutes. The viscosity is given in Pa·s, at a given temperature and time.

The viscosity is in reality measured at a stage where the particles are not yet formed, i.e. on the oil, gelling agent and active substance premix, as emerges on reading the detailed process below.

The choice of the oil(s) included in the formulation of the oily phase and also that of the gelling polymer(s) may be adjusted by a person skilled in the art such that the structured oily phase of the particles in accordance with the invention satisfies the melting point and viscosity criteria described above.

Gelling Polymer

The calibrated and spherical oily particles are advantageously obtained from at least one oily phase structured with at least one gelling polymer chosen especially from a semi-crystalline polymer, a polysaccharide monoalkyl or polyalkyl ester, a polyamide, a silicone polyamide or a diblock, triblock, multiblock and/or radial-block copolymer, and mixtures thereof.

For the purposes of the present invention, the term “polymer” is intended to denote compounds comprising at least two repeating units, preferably at least three repeating units and especially at least 10 repeating units.

The content of gelling polymer(s) in the particles in accordance with the invention may range from 1% to 80% by weight or even from 1% to 60% by weight relative to the total weight of the particle.

Advantageously, the weight ratio between the gelling polymer and the oily phase of the particle according to the invention may range from 0.01 to 4, or even from 0.05 to 2 and in particular from 0.1 to 1.

Semi-Crystalline Polymer

For the purposes of the invention, the term “semi-crystalline polymer” means polymers comprising a crystallizable portion, side chain or block in the skeleton, and an amorphous portion in the skeleton and having a first-order reversible change of phase temperature, in particular of melting (solid-liquid transition). When the crystallizable portion is a block of the polymer skeleton, this crystallizable block has a different chemical nature from that of the amorphous blocks; in this case, the semi-crystalline polymer is a block polymer, for example of the diblock, triblock or multiblock type.

The semi-crystalline polymers that may be used for the implementation of the present invention are solid at room temperature and preferably have a melting point (or gel point) of less than 80° C.

They comprise:

a) a polymer skeleton, and

b) at least one crystallizable organic side chain and/or one crystallizable organic block forming part of the skeleton of the said polymer, the said polymer having a number-average molecular mass of greater than or equal to 2000.

Advantageously, the semi-crystalline polymer(s) of the composition of the invention have a number-average molecular mass Mn of greater than or equal to 2000, for example ranging from 2000 to 800 000 and especially from 3000 to 500 000.

According to one embodiment, the semi-crystalline polymers that may be used in the context of the invention have a melting point m.p. of less than 70° C. and especially of less than 50° C. The semi-crystalline polymer advantageously has a melting point m.p. in the range from 40° C. to less than 80° C. In reality, the semi-crystalline polymer may be a mixture of semi-crystalline polymers. In this case, it is the mixture that has a melting point m.p. within the said range. In other words, the mixture may comprise a semi-crystalline polymer having a melting point outside this range, provided that the mixture itself has a melting point within the said range. The melting point may be measured especially by any known method and in particular with a differential scanning calorimeter (DSC).

According to one embodiment variant, the crystallizable blocks or chains of the semi-crystalline polymers represent at least 30% or even at least 40% of the total weight of each polymer. The semi-crystalline polymers of the invention containing crystallizable blocks may be block or multiblock polymers. They may be obtained by polymerization of monomers containing reactive double bonds (ethylenic bonds) or by polycondensation. When the polymers of the invention are polymers containing crystallizable side chains, they are advantageously in random or statistical form.

The semi-crystalline polymers that may be used in the invention are, for example:

1. block copolymers of polyolefins of controlled crystallization, whose monomers are described in document EP-A-0 951 897,

2. polycondensates, especially of aliphatic or aromatic polyester type or of aliphatic/aromatic copolyester type,

3. homopolymers or copolymers bearing at least one crystallizable side chain and homopolymers or copolymers bearing at least one crystallizable block in the skeleton, for instance those described in patent U.S. Pat. No. 5,156,911,

4. homopolymers or copolymers bearing at least one crystallizable side chain, bearing fluoro group(s) patent application such as those described in WO-A-01/19333,

5. and mixtures thereof.

In the last two cases (3 and 4), the crystallizable side chain(s) or block(s) is (are) hydrophobic.

The crystalline polymers containing crystallizable side chains, or bearing in the skeleton at least one crystallizable block suitable for use in the invention, are, for example, described below.

A) Semi-Crystalline Polymers Containing Crystallizable Side Chains

Mention may be made in particular of the polymers defined in documents U.S. Pat. No. 5,156,911 and WO-A-01/19333. They are homopolymers or copolymers comprising from 50% to 100% by weight of units resulting from the polymerization of one or more monomers bearing (a) crystallizable hydrophobic side chain(s).

These homopolymers or copolymers are of any nature, provided that they meet the conditions mentioned hereinbelow with, in particular, the characteristic of being soluble or dispersible in the oily phase, by heating above their melting point mp (or gel point). They can result:
from the polymerization, especially the free-radical polymerization, of one or more monomers containing (a) reactive or ethylenic double bond(s) with respect to a polymerization, namely a vinyl, (meth)acrylic or allylic group,
from the polycondensation of one or more monomers bearing co-reactive groups (carboxylic acid, sulfonic acid, alcohol, amine or isocyanate), such as, for example, polyesters, polyurethanes, polyethers, polyureas or polyamides.

In general, the crystallizable units (chains or blocks) of semi-crystalline polymers that can be used in the context of the invention are derived from monomer(s) containing (a) crystallizable block(s) or chain(s), used for manufacturing semi-crystalline polymers. These polymers are chosen especially from homopolymers and copolymers resulting from the polymerization of at least one monomer containing (a) crystallizable chain(s) that may be represented by formula X: 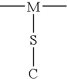
in which M represents an atom of the polymer skeleton, S represents a spacer and C represents a crystallizable group.

The crystallizable chains “—S—C” may be aliphatic or aromatic, and optionally fluorinated or perfluorinated. “S” especially represents a group (CH₂)ₙ or (CH₂CH₂O)ₙ or (CH₂O), which may be linear or branched or cyclic, with n being an integer ranging from 0 to 22. Preferably, “S” is a linear group. Preferably, “S” and “C” are different.

When the crystallizable chains are aliphatic chains, they comprise at least 11 carbon atoms and not more than 40 carbon atoms and better still not more than 24 carbon atoms. They are especially, for example, alkyl chains containing at least 12 carbon atoms, and they can be alkyl chains containing from 14 to 24 carbon atoms C₁₄-C₂₄. They can be hydrocarbon-based alkyl chains (carbon and hydrogen atoms) or fluoroalkyl or perfluoroalkyl chains (carbon atoms, fluorine atoms and optionally hydrogen atoms). When they are fluoroalkyl or perfluoroalkyl chains, they contain at least 11 carbon atoms, at least 6 of which carbon atoms are fluorinated.

As examples of semi-crystalline polymers or copolymers containing (a) crystallizable chain(s), mention may be made of those resulting from the polymerization of at least one monomer with a crystallizable chain chosen from (meth)acrylates of saturated C₁₄-C₂₄ alkyls (C₁₄-C₂₄ means that the alkyl group contains from 14 to 24 carbon atoms); C₁₁-C₁₅ perfluoroalkyl (meth)acrylates (alkyl group containing 11 to 15 carbon atoms); C₁₄ to C₂₄ N-alkyl(meth)acrylamides with or without a fluorine atom; vinyl esters containing C₁₄ to C₂₄ alkyl or perfluoroalkyl chains, with a perfluoroalkyl chain containing at least 6 fluorine atoms; vinyl ethers containing C₁₄ to C₂₄ alkyl or perfluoroalkyl chains, with a perfluoroalkyl chain containing at least 6 fluorine atoms; C₁₄ to C₂₄ α-olefins, for instance octadecene; C₁₄ to C₂₄ para-alkylstyrenes, and mixtures thereof.

For the purposes of the invention, the term “alkyl” means a saturated group especially containing from 8 to 24 carbon atoms (C₈ to C₂₄), except where otherwise mentioned.

When the polymers result from a polycondensation, the hydrocarbon-based and/or fluorinated crystallizable chains as defined above are borne by a monomer that may be a diacid, a diol, a diamine or a diisocyanate.

When the polymers used in the composition of the invention are copolymers, they additionally contain from 0% to 50% of groups Y or Z resulting from copolymerization:

α) with Y which is a polar or non-polar monomer or a mixture of the two:
when Y is a polar monomer, it is either a monomer bearing polyoxyalkylenated groups (especially oxyethylenated and/or oxypropylenated groups), a hydroxyalkyl (meth)acrylate, for instance hydroxyethyl acrylate, (meth)acrylamide, an N-alkyl(meth)acrylamide, an N,N-dialkyl(meth)acrylamide such as, for example, N,N-diisopropylacrylamide or N-vinylpyrrolidone (NVP), N-vinylcaprolactam, or a monomer bearing at least one carboxylic acid group, for instance (meth)acrylic acid, crotonic acid, itaconic acid, maleic acid or fumaric acid, or bearing a carboxylic acid anhydride group, for instance maleic anhydride, and mixtures thereof.
when Y is a non-polar monomer, it may be an ester of the linear, branched or cyclic alkyl (meth)acrylate type, a vinyl ester, an alkyl vinyl ether, an α-olefin, styrene or styrene substituted with an alkyl group containing from 1 to 10 carbon atoms (C₁ to C₁₀), for instance α-methylstyrene, or a macromonomer of the polyorganosiloxane type containing vinyl unsaturation.

β) with Z which is a polar monomer or a mixture of polar monomers, Z having the same definition as the “polar Y” defined above.

Preferably, the semi-crystalline polymers containing a crystallizable side chain are alkyl (meth)acrylate or alkyl(meth)acrylamide homopolymers with an alkyl group as defined above, and especially of C₁₄-C₂₄, copolymers of these monomers with a hydrophilic monomer preferably of different nature from (meth)acrylic acid, and mixtures thereof. The copolymers can be, for example, alkyl methacrylate copolymers or copolymers of alkylmethacrylamide containing a C₁₄ to C₂₄ alkyl group with N-vinylpyrrolidone or hydroxyethyl (meth)acrylate, and mixtures thereof.

B) Polymers bearing at least one crystallizable block in the skeleton

This is also a case of polymers that are soluble or dispersible in the oil or oily phase by heating above their melting point mp. These polymers are especially block copolymers consisting of at least 2 blocks of different chemical nature, one of which is crystallizable.

As polymers bearing in the skeleton at least one crystallizable block that are suitable for use in the invention, mention may be made of:

1. the polymers defined in document U.S. Pat. No. 5,156,911;

2. block copolymers of olefin or of cycloolefin containing a crystallizable chain, for instance those derived from the block polymerization of:
cyclobutene, cyclohexene, cyclooctene, norbornene (i.e. bicyclo(2,2,1)-2-heptene), 5-methylnorbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinylnorbornene, 1,4,5,8-dimethano-1,2,3,4,4a,5,8a-octahydronaphthalene, dicyclopentadiene, or mixtures thereof,
with ethylene, propylene, 1-butene, 3-methyl-1-butene, 1-hexene, 4-methyl-1-pentene, 1-octene, 1-decene or 1-eicosene, or mixtures thereof. These block copolymers may be in particular (ethylene/norbornene) block copolymers and (ethylene/propylene/ethylidenenorbornene) block terpolymers.

Those resulting from the block copolymerization of at least 2 C₂-C₁₆, better still C₂-C₁₂ α-olefins such as those mentioned above and in particular block bipolymers of ethylene and of 1-octene may also be used.

3. copolymers containing at least one crystallizable block, the rest of the copolymer being amorphous (at room temperature). These copolymers may also contain two crystallizable blocks of different chemical nature. The preferred copolymers are those that simultaneously contain at room temperature a crystallizable block and an amorphous block that are both hydrophobic and lipophilic, sequentially distributed; mention may be made, for example, of polymers containing one of the crystallizable blocks and one of the amorphous blocks below:
block that is crystallizable by nature: a) polyester for instance poly(alkylene terephthalate), b) polyolefin, for instance polyethylenes or polypropylenes.
amorphous and lipophilic block, for instance amorphous polyolefins or copoly(olefin)s such as poly(isobutylene), hydrogenated polybutadiene or hydrogenated poly(isoprene).

As examples of such copolymers containing a crystallizable block and an amorphous block, mention may be made of:
α) poly(ε-caprolactone)-b-poly(butadiene) block copolymers, preferably used hydrogenated, such as those described in the article “Melting behaviour of poly(ε-caprolactone)-block-polybutadiene copolymers” from S. Nojima, Macromolecules, 32, 3727-3734 (1999),
β) the hydrogenated block or multiblock poly(butylene terephthalate)-b-poly(isoprene) block copolymers cited in the article “Study of morphological and mechanical properties of PP/PBT” by B. Boutevin et al., Polymer Bulletin, 34, 117-123 (1995),
γ) the poly(ethylene)-b-copoly(ethylene/propylene) block copolymers cited in the articles “Morphology of semi-crystalline block copolymers of ethylene-(ethylene-alt-propylene)” by P. Rangarajan et al., Macromolecules, 26, 4640-4645 (1993) and “Polymer aggregates with crystalline cores: the system poly(ethylene)-poly(ethylene-propylene)” by P. Richter et al., Macromolecules, 30, 1053-1068 25 (1997),
δ) the poly(ethylene)-b-poly(ethylethylene) block copolymers cited in the general article “Crystallization in block copolymers” by I. W. Hamley, Advances in Polymer Science, Vol. 148, 113-137 (1999).

The semi-crystalline polymers that may be used in the context of the invention may be non-crosslinked or partially crosslinked, provided that the degree of crosslinking does not impede their dissolution or dispersion in the liquid oily phase by heating above their melting point. It may then be a case of chemical crosslinking, by reaction with a multifunctional monomer during the polymerization. It may also be a case of physical crosslinking, which may then be due either to the establishment of bonds of hydrogen or dipolar type between groups borne by the polymer, for instance dipolar interactions between carboxylate ionomers, these interactions being in small amount and borne by the polymer skeleton; or due to a phase separation between the crystallizable blocks and the amorphous blocks borne by the polymer.

Preferably, the semi-crystalline polymers that are suitable for the invention are non-crosslinked.

As particular examples of semi-crystalline polymers that may be used in the composition according to the invention, mention may be made of the Intelimer® products from the company Landec described in the brochure “Intelimer® Polymers”. These polymers are in solid form at room temperature (25° C.). They bear crystallizable side chains and contain the monomer as defined in formula X above. Mention may be made especially of “Landec IP22®”, with a melting point m.p. of 56° C., which is a viscous, impermeable, non-tacky product at room temperature.

It is also possible to use the polymer “Structure O” sold by the company National Starch, such as the product described in document U.S. Pat. No. 5,736,125, of m.p. 44° C., and also semi-crystalline polymers containing crystallizable side chains comprising fluoro groups as described in Examples 1, 4, 6, 7 and 8 of document WO-A-01/19333.

It is also possible to use the semi-crystalline polymers obtained by copolymerization of stearyl acrylate and of acrylic acid or of NVP, or by copolymerization of behenyl acrylate and of acrylic acid or NVP, as described in document U.S. Pat. No. 5,519,063 or EP-A-0 550 745.

According to one particular embodiment variant, the semi-crystalline polymers that are suitable for use in the present invention are especially alkyl acrylates, among which mention may be made of the Landec copolymers:

Doresco IPA 13-1®: polystearyl acrylate, m.p. of 49° C. and MW of 145 000;

Doresco IPA 13-3®: polyacrylate/methacrylic acid, m.p. of 65° C. and MW of 114 000;

Doresco IPA 13-4®: polyacrylate/vinylpyrrolidone, m.p. of 44° C. and MW of 387 000;

Doresco IPA 13-5®: polyacrylate/hydroxyethyl methacrylate, m.p. of 47° C. and MW of 397 600;

Doresco IPA 13-6®: polybehenyl acrylate, m.p. of 66° C.

Polyamides

The polyamides that may advantageously be used in the preparation of the particles according to the invention are especially those described in document U.S. Pat. No. 5,783,657 from the company Union Camp.

The polyamides that are suitable for use in the invention especially satisfy the following formula: 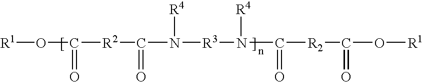
in which:
n is a whole number of amide units such that the number of ester groups represents from 10% to 50% of the total number of ester and amide groups;
R¹ is in each instance, independently, an alkyl or alkenyl group containing at least 4 carbon atoms and especially from 4 to 24 carbon atoms;
R² represents in each instance, independently, a C₄ to C₅₅ hydrocarbon-based group, on condition that at least 50% of the groups R² represent a C₃₀ to C₅₅ hydrocarbon-based group;
R³ represents in each instance, independently, an organic group containing at least 2 carbon atoms, hydrogen atoms and optionally one or more oxygen or nitrogen atoms; and
R⁴ represents in each instance, independently, a hydrogen atom, a C₁ to C₁₀ alkyl group or a direct bond to R³ or to another R⁴ such that the nitrogen atom to which both R³ and R⁴ are attached forms part of a heterocyclic structure defined by R⁴—N—R³, with at least 50% of the groups R⁴ representing a hydrogen atom.

According to one embodiment variant, the ester groups of these polyamides represent from 15% to 40% and at best from 20% to 35% of the total number of ester and amide groups. Furthermore, n advantageously represents an integer ranging from 1 to 10 and better still from 1 to 5.

R¹ is especially a C₁₂ to C₂₂ or even C₁₆ to C₂₂ alkyl group. R² may especially be a C₁₀ to C₄₂ hydrocarbon-based (alkylene) group. In particular, at least 50% and better still at least 75% of the groups R² may be groups containing from 30 to 42 carbon atoms. The other groups R² are C₄ to C₁₉ and in particular C₄ to C₁₂ hydrogen-containing groups. R³ may represent a C₂ to C₃₆ hydrocarbon-based group or a polyoxyalkylene group and R⁴ represents a hydrogen atom. In particular, R³ may represent a C₂ to C₁₂ hydrocarbon-based group. The hydrocarbon-based groups may be linear, cyclic or branched, and saturated or unsaturated groups. Moreover, the alkyl and alkylene groups may be linear or branched, and saturated or unsaturated groups.

As examples of structuring polyamides that may be used in the invention, mention may also be made of polyamide resins resulting from the condensation of an aliphatic dicarboxylic acid and of a diamine (including compounds containing more than two carbonyl groups and two amine groups), the carbonyl and amine groups of adjacent individual units being condensed via an amide bond. These polyamide resins are especially the products sold under the brand name Versamid by the companies General Mills, Inc. and Henkel Corp., under the brand name Onamid, especially Onamid S or C. These resins have a weight-average molecular mass ranging from. 6000 to 9000. Documents U.S. Pat. No. 3,645,705 and U.S. Pat. No. 3,148,125 describe these resins. According to one embodiment variant, Versamid 930 or 744 is used.

It is also possible to use the polyamides sold or manufactured by the company Arizona under the references Uni-Rez (2658, 2931, 2970, 2621, 2613, 2624, 2665, 1554, 2623, 2662) and the product sold under the reference Macromelt 6212® by the company Henkel. Patent U.S. Pat. No. 5,500,209 describes polymers of this type.

As examples of structuring polyamides that may be used in the composition according to the invention, mention may also be made of the commercial products sold or manufactured by the company Arizona Chemical under the names Uniclear 80® and Uniclear 100°. They are sold, respectively, in the form of an 80% (active material) gel and a 100% (active material) gel in a mineral oil. They have a softening point of from 88 to 105° C. These commercial products are a mixture of copolymer of a C₃₆ diacid condensed with ethylenediamine, with an average molecular mass of about 6000. The ester end groups result from the esterification of the remaining acid end groups with cetyl or stearyl alcohol or mixtures thereof (also known as cetylstearyl alcohol).

The structuring polyamides advantageously have a softening point of greater than 60° C., which may be up to 190° C. They preferably have a softening point of less than 150° C., ranging from 70 to 130° C. and better still from 80 to 105° C.

The structuring of the oily phase may be obtained by means of one or more polyamides defined above. In general, these polyamides are in the form of mixtures, these mixtures also possibly containing a synthetic product corresponding to a polyamide as defined above with n being 0, i.e. a diester.

The polyamides used in the present invention have, on account of their fatty chain, good solubility in the oily phase and thus lead to compositions that are macroscopically homogeneous, even with a high polymer content.

As examples of polyamides that are suitable for use in the present invention, mention may be made of the copolymer of ethylenediamine/stearyl dimerdilinoleate, sold under the reference Uniclear 100® VG by the company Arizona Chemical.

Silicone Polyamides

The polymers (homopolymers or copolymers) of polyamide type that are suitable for use in the invention have an average molecular mass included in the range from 500 to 500 000 and contain at least one group comprising:

at least one polyorganosiloxane group, comprising from 1 to 1000 organosiloxane units, in the chain of the group or in the form of a graft, and

at least two groups capable of establishing hydrogen interactions, chosen from ester, amide, sulfonamide, carbamate, thiocarabamate, urea, thiourea, oxamido, guanidino and biguanidino groups, and combinations thereof, on condition that at least one of these groups is other than an ester group,

the polymer being solid at room temperature and soluble in the oily phase at a temperature ranging from 25 to 150° C. In particular, the polymer is soluble in the oily phase at a temperature ranging from 41 to 120° C.

The polymers that are suitable for use in the invention, and used as oil-gelling agent, may belong to the following two families:

polyorganosiloxanes comprising at least two groups capable of establishing hydrogen interactions, these two groups being located in the polymer chain, and/or

polyorganosiloxanes comprising at least two groups capable of establishing hydrogen interactions, these two groups being located on grafts or branches.

The polymers comprising two groups capable of establishing hydrogen interactions in the polymer chain may be polymers comprising at least one unit corresponding to the first formula below: 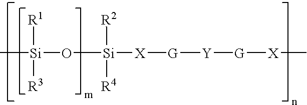
in which:

1. R¹, R², R³ and R⁴, which may be identical or different, represent a group chosen from:

linear, branched or cyclic, saturated or unsaturated C₁ to C₄₀ hydrocarbon-based groups, which may contain in their chain one or more oxygen, sulfur and/or nitrogen atoms, and which may be partially or totally substituted with fluorine atoms,

C₆ to C₁₀ aryl groups optionally substituted with one or more C₁ to C₄ alkyl groups,

polyorganosiloxane chains optionally containing one or more oxygen, sulfur and/or nitrogen atoms;

2. The groups X, which may be identical or different, represent a linear or branched C₁ to C₃₀ alkylenediyl group, which may contain in its chain one or more oxygen and/or nitrogen atoms;

3. Y is a linear or branched alkylene, arylene, cycloalkylene, alkylarylene or arylalkylene, saturated or unsaturated C₁ to C₅₀ divalent group, which may comprise one or more oxygen, sulfur and/or nitrogen atoms, and/or bear as substituent one of the following atoms or groups of atoms: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl groups, C₁ to C₃ hydroxyalkyl groups and C₁ to C₆ aminoalkyl groups, or;

4. Y represents a group corresponding to the formula 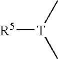
in which:

T represents a linear or branched, saturated or unsaturated C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and

R⁵ represents a linear or branched C₁ to C₅₀ alkyl group, or a polyorganosiloxane chain, possibly comprising one or more ester, amide, urethane, thiocarbamate, urea, thiourea and/or sulfonamide groups which may or may not be linked to another chain of the polymer,

5. the groups G, which may be identical or different, represent divalent groups chosen from: 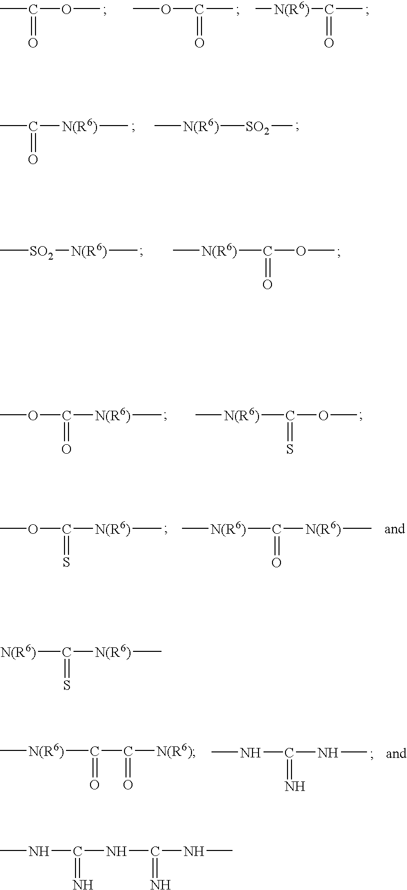
in which R⁶ represents a hydrogen atom or a linear or branched C₁ to C₂₀ alkyl group.

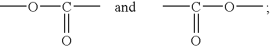

6. n is an integer ranging from 2 to 500 and in particular from 2 to 200, and m is an integer ranging from 1 to 1000, in particular from 1 to 700 and better still from 6 to 200.

According to one embodiment variant, 80% of the groups R¹, R², R³ and R⁴ of the polymer may be chosen especially from methyl, ethyl, phenyl and 3,3,3-trifluoropropyl groups.

According to another embodiment variant, Y may represent various divalent groups, optionally also comprising one or two free valencies to establish bonds with other units of the polymer or copolymer. Y may especially represent a group chosen from:

a. linear C₁ to C₂₀ and especially C₁ to C₁₀ alkylene groups;

b. C₃₀ to C₅₆ branched alkylene groups possibly comprising rings and unconjugated unsaturations;

c. C₅-C₆ cycloalkylene groups;

d. phenylene groups optionally substituted with one or more C₁ to C₄₀ alkyl groups;

e. C₁ to C₂₀ alkylene groups, comprising from 1 to 5 amide groups;

f. C₁ to C₂₀ alkylene groups, comprising one or more substituents, chosen from hydroxyl, C₃ to C₈ cycloalkane, C₁ to C₃ hydroxyalkyl and C¹ to C₆ alkylamine groups;

g. polyorganosiloxane chains of formula: 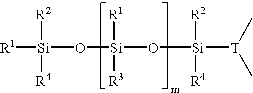
in which R¹, R², R³ and R⁴, T and m are as defined above; and

h. polyorganosiloxane chains of formula:

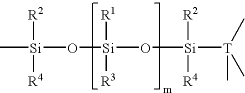

The polyorganosiloxanes of the second family may be polymers comprising at least one unit corresponding to the second formula below: 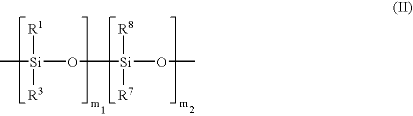
in which:

R¹ and R³, which may be identical or different, are as defined above for the preceding formula;

R⁷ represents a group as defined above for R¹ and R³, or represents the group of formula —X-G-R⁹ in which X and G are as defined above for the preceding formula and R⁹ represents a hydrogen atom or a linear, branched or cyclic, saturated or unsaturated C₁ to C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups;

R⁸ represents a group of formula —X-G-R⁹ in which X, G and R⁹ are as defined above;

m₁ is an integer ranging from 1 to 998; and

m₂ is an integer ranging from 2 to 500.

According to the invention, the silicone polyamide used as gelling agent may be a homopolymer, i.e. a polymer comprising several identical units, in particular units according to the formulae defined above.

According to the invention, it is also possible to use a silicone polyamide consisting of a copolymer comprising several different units according to the first formula above, i.e. a polymer in which at least one of the groups R¹, R², R³, R⁴, X, G, Y, m and n is different in one of the units. The copolymer may also be formed from several units according to the second formula above, in which at least one of the groups R¹, R³, R⁷, R⁸, m₁ and m₂ is different in at least one of the units.

It is also possible to use a copolymer comprising at least one unit according to the first formula and at least one unit according to the second formula, the units according to the first formula and the units according to the second formula possibly being identical to or different from each other.

According to one variant of the invention, it is also possible to use a silicone polyamide of copolymer type also comprising at least one hydrocarbon-based unit comprising two groups capable of establishing hydrogen interactions chosen from ester, amide, sulfonamide, carbamate, thiocarbamate, urea and thiourea groups, and combinations thereof. These copolymers may be block copolymers, sequenced copolymers or grafted copolymers.

According to one embodiment variant, the groups capable of establishing hydrogen interactions are amide groups of formulae —C(O)NH— and —HN—C(O)—. In this case, the gelling agent may be, for example, a polymer comprising at least one unit according to the third or fourth formula below: 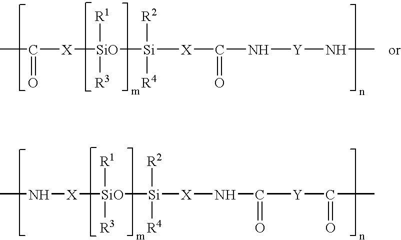
in which R¹, R², R³, R⁴, X, Y, m and n are as defined above.

Such a unit may be obtained:

either via a condensation reaction between a silicone containing α,ω-carboxylic acid end groups and one or more diamines, according to the following reaction scheme: 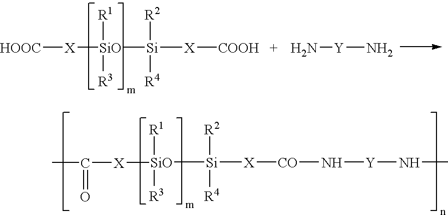
or via reaction of two α-unsaturated carboxylic acid molecules with a diamine according to the following reaction scheme: CH₂═CH—X¹—COOH+H₂N—Y—NH₂->CH₂═CH—X¹—CO—NH—Y—NH—CO—X¹—CH═CH₂ followed by addition of a siloxane to the ethylenic unsaturations, according to the following scheme: CH₂═CH—X¹—CO—NH—Y—NH—CO—X¹—CH═CH₂+ 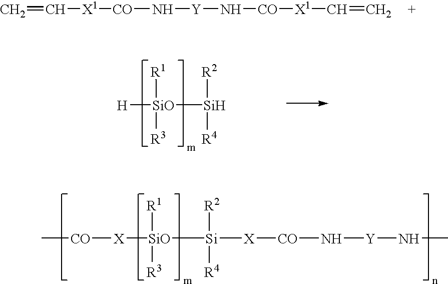
in which X¹—(CH₂)₂— corresponds to X defined above and Y, R¹, R², R³, R⁴ and m are as defined above;

or via reaction with a silicone containing α,ω-NH₂ end groups and of a diacid of formula HOOC—Y—COOH according to the following reaction scheme:

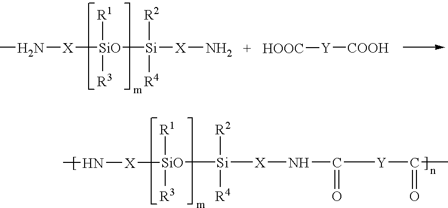

In the polyamides according to the third and fourth formulae presented above:

m is especially in the range from 1 to 700, or even from 15 to 500 and better still from 15 to 45, and

n is in particular in the range from 1 to 500, especially from 1 to 100 and better still from 4 to 25,

X is especially a linear or branched alkylene chain containing from 1 to 30 carbon atoms and in particular 3 to 10 carbon atoms, and

Y is especially a linear or branched alkylene chain or a chain that may comprise rings and/or unsaturations, containing from 1 to 40 carbon atoms, in particular from 1 to 20 carbon atoms and better still from 2 to 6 carbon atoms, in particular 6 carbon atoms.

In the third and fourth formulae presented above, the alkylene group representing X or Y may optionally contain in its alkylene part at least one of the following elements:

1) 1 to 5 amide, urea or carbamate groups,

2) a C₅ or C₆ cycloalkyl group, and

3) a phenylene group optionally substituted with 1 to 3 identical or different C₁ to C₃ alkyl groups.

In the third and fourth formulae presented above, the alkylene groups may also be substituted with at least one element chosen from the group consisting of:

a hydroxyl group,

a C₃ to C₈ cycloalkyl group,

one to three C₁ to C₄₀ alkyl groups,

a phenyl group optionally substituted with one to three C₁ to C₃ alkyl groups,

a C₁ to C₃ hydroxyalkyl group, and

a C₁ to C₆ aminoalkyl group. In the third and fourth formulae presented above, Y may also represent: 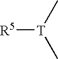
in which R⁵ represents a polyorganosiloxane chain, and T represents a group of formula: 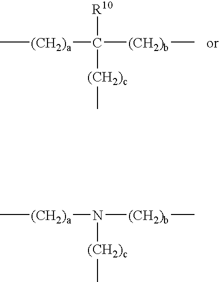
in which a, b and c are, independently, integers ranging from 1 to 10, and R¹⁰ is a hydrogen atom or a group such as those defined for R¹, R², R³ and R⁴.

In the third and fourth formulae presented above, R¹, R², R³ and R⁴ especially represent, independently, a linear or branched C₁ to C₄₀ alkyl group, in particular a CH₃, C₂H₅, n-C₃H₇ or isopropyl group, a polyorganosiloxane chain or a phenyl group optionally substituted with one to three methyl or ethyl groups.

As has been seen previously, the polymer may also comprise identical or different units according to the third or fourth formula presented above.

Thus, the polymer may be a silicone polyamide containing several units according to the third or fourth formula presented above, of different lengths, or a polyamide corresponding to the fifth formula below: 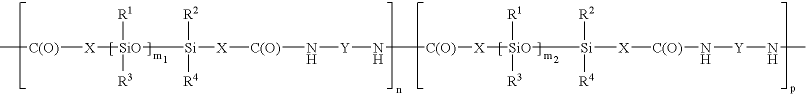
in which X, Y, n, R¹ to R⁴ have the meanings given above, m₁ and m₂, which are different, are chosen in the range from 1 to 1000, and p is an integer ranging from 2 to 300.

In this formula, the units may be structured to form either a block copolymer or a random copolymer or an alternating copolymer. In this copolymer, the units may be not only of different lengths but also of different chemical structures, for example having different groups Y. In this case, the copolymer may correspond to the sixth formula: 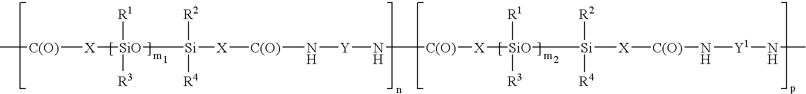
in which R¹ to R⁴, X, Y, m₁, m₂, n and p have the meanings given above and Y¹ is different from Y, but is chosen from the groups defined for Y. As previously, the various units may be structured to form either a block copolymer or a random copolymer or an alternating copolymer.

According to one embodiment of the invention, the gelling silicone polyamide may also consist of a grafted copolymer. Thus, the polyamide containing silicone units may be grafted and optionally crosslinked with silicone chains containing amide groups. Such polymers may be synthesized with trifunctional amines.

In this case, the copolymer may comprise at least one unit according to the seventh formula below: 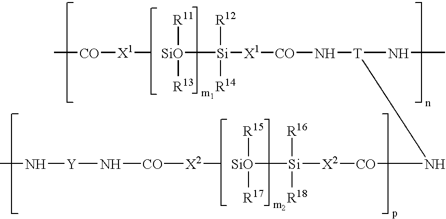
in which X¹ and X², which may be identical or different, have the meaning given for X in the first formula above, n is as defined in the first formula above, Y and T are as defined in the first formula above, R¹¹ to R¹⁸ are groups chosen from the same group as the groups R¹ to R⁴, m₁ and m₂ are numbers in the range from 1 to 1000, and p is an integer ranging from 2 to 500.

In the seventh formula presented above, in particular:

p is in the range from 1 to 25 and better still from 1 to 7,

R¹¹ to R¹⁸ are methyl groups,

T corresponds to one of the following formulae: 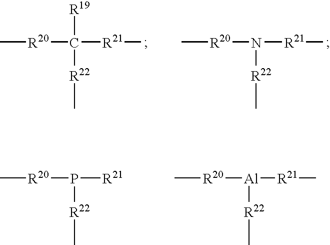
in which R¹⁹ is a hydrogen atom or a group chosen from the groups defined for R¹ to R⁴, and R²⁰, R²¹ and R²² are, independently, linear or branched alkylene groups,

T preferably corresponds in particular to the formula: 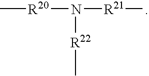
with, especially, R²⁰, R²¹ and R²² representing —CH₂—CH₂—,

m₁ and m₂ are in the range from 15 to 500 or even from 15 to 45,

—X¹ and X² represent —(CH₂)₁₀—, and

Y represents —CH₂—.

These polyamides containing a grafted silicone unit according to the seventh formula presented above may be copolymerized with silicone polyamides according to the second formula to form block copolymers, alternating copolymers or random copolymers. The weight percentage of grafted silicone units according to the seventh formula in the copolymer may range from 0.5% to 30% by weight.

According to one embodiment, the siloxane units may be in the main chain or skeleton of the polymer, but they may also be present in grafted chains or side chains. In the main chain, the siloxane units may be in the form of segments as described above. In the side chains or grafted chains, the siloxane units may appear individually or in segments.

According to one embodiment of the invention, the siloxane-based polyamides may especially be:

polyamides according to the third formula presented above in which m is from 15 to 50;

mixtures of two or more polyamides in which at least one polyamide has a value of m in the range from 15 to 50 and at least one polyamide has a value of m in the range from 30 to 50; polymers according to the fifth formula described above with m₁ chosen in the range from 15 to 50 and m₂ chosen in the range from 30 to 500 with the part corresponding to m₁ representing 1% to 99% by weight relative to the total weight of the polyamide and the part corresponding to m₂ representing 1% to 99% by weight relative to the total weight of the polyamide;

polyamide blends according to the third formula described above, combining:

1. 80% to 99% by weight of a polyamide in which n is equal to 2 to 10 and in particular 3 to 6, and

2. 1% to 20% of a polyamide in which n is in the range from 5 to 500 and in particular from 6 to 100;

polyamides corresponding to the sixth formula presented above in which at least one of the groups Y and Y¹ contains at least one hydroxyl substituent;

polyamides according to the third formula synthesized with at least part of an activated diacid (diacid chloride, dianhydride or diester) instead of the diacid;

polyamides according to the third formula in which X represents —(CH₂)₃— or —(CH₂)₁₀—; and

polyamides according to the third formula in which the polyamides end with a monofunctional chain chosen from the group consisting of monofunctional amines, monofunctional acids, monofunctional alcohols, including fatty acids, fatty alcohols and fatty amines, for instance octylamine, octanol, stearic acid and stearyl alcohol.

According to one embodiment of the invention, the ends of the polymer chains may end with:

a C₁ to C₅₀ alkyl ester group by introducing during the synthesis a C₁ to C₅₀ mono alcohol,

a C₁ to C₅₀ alkylamide group by taking as stopper a monoacid if the silicone contains α,ω-diamino, or a monoamine if the silicone contains α,ω-dicarboxylic acid.

According to one embodiment variant of the invention, it is possible to use a copolymer of silicone polyamide and of hydrocarbon-based polyamide, i.e. a copolymer comprising units according to the third or fourth formula and hydrocarbon-based polyamide units. In this case, the polyamide-silicone units may be located at the ends of the hydrocarbon-based polyamide.

Polyamide-based gelling agents containing silicones may be produced by silyl amidation of polyamides based on fatty acid dimer. This approach involves the reaction of free acid sites existing on a polyamide as end sites, with oligosiloxane-monoamines and/or oligosiloxane-diamines (amidation reaction), or alternatively with oligosiloxane alcohols or oligosiloxane diols (esterification reaction). The esterification reaction requires the presence of acid catalysts, as is known in the art. It is desirable for the polyamide containing free acid sites, used for the amidation or esterification reaction, to have a relatively high number of acid end groups (for example polyamides with high acid numbers, for example from 15 to 20).

For the amidation of the free acid sites of the hydrocarbon-based polyamides, siloxane diamines with 1 to 300, more particularly 2 to 50 and better still 2, 6, 9.5, 12, 13.5, 23 or 31 siloxane groups may be used for the reaction with hydrocarbon-based polyamides based on fatty acid dimers. Siloxane diamines containing 13.5 siloxane groups are preferred, and the best results are obtained with the siloxane-diamine containing 13.5 siloxane groups and polyamides with high numbers of carboxylic acid end groups.

The reactions may be performed in xylene to extract the water produced from the solution by azeotropic distillation, or at higher temperatures (about 180 to 200° C.) without solvent. Typically, the amidation efficacy and the reaction rates decrease when the siloxane diamine is longer, i.e. when the number of siloxane groups is higher. Free amine sites may be blocked after the initial amidation reaction of the diaminosiloxanes by reacting them either with an acidic siloxane or an organic acid such as benzoic acid.

For the esterification of the free acid sites on the polyamides, this may be performed in boiling xylene with about 1% by weight, relative to the total weight of the reagents, of para-toluenesulfonic acid as catalyst.

These reactions performed on the carboxylic acid end groups of the polyamide lead to the incorporation of silicone units only at the ends of the polymer chain.

As an example of a gelling polymer of silicone polyamide type that is suitable for use in the invention, mention may be made of the polyamide/polydimethylsiloxane block copolymer sold, for example, under the reference DC2-8178 Gellant by the company Dow Corning.

Polysaccharide Monoalkyl or Polyalkyl Esters

Among the saccharide or polysaccharide monoalkyl or polyalkyl esters that are suitable for use in the invention, mention may be made of dextrin or inulin alkyl or polyalkyl esters.

It may especially be a dextrin mono- or polyester of at least one fatty acid corresponding especially to the following formula: 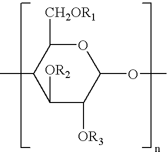
in which:
n is an integer ranging from 3 to 200, especially ranging from 20 to 150 and in particular ranging from 25 to 50,
the radicals R₁, R₂ and R₃, which may be identical or different, are chosen from hydrogen and an acyl group (R—CO—) in which the radical R is a linear or branched, saturated or unsaturated hydrocarbon-based group containing from 7 to 29, in particular from 7 to 21, especially from 11 to 19, more particularly from 13 to 17, or even 15, carbon atoms, with the proviso that at least one of the said radicals R₁, R₂ or R₃ is other than hydrogen.

In particular, R₁, R₂ and R₃ may represent hydrogen or an acyl group (R—CO—) in which R is a hydrocarbon-based radical as defined above, with the proviso that at least two of the said radicals R₁, R₂ or R₃ are identical and other than hydrogen.

The radicals R₁, R₂ and R₃ may all contain an acyl group (R—CO), which is identical or different and especially identical.

In particular, n mentioned above advantageously ranges from 25 to 50 and is especially equal to 38 in the general formula of the saccharide ester that may be used in the present invention.

When the radicals R₁, R₂ and/or R₃, which may be identical or different, contain an acyl group (R—CO), these radicals may be chosen especially from caprylic, capric, lauric, myristic, palmitic, stearic, arachic, behenic, isobutyric, isovaleric, 2-ethylbutyric, ethylmethylacetic, isoheptanoic, 2-ethylhexanoic, isononanoic, isodecanoic, isotridecanoic, isomyristic, isopalmitic, isostearic, isoarachic, isohexanoic, decenoic, dodecenoic, tetradecenoic, myristoleic, hexadecenoic, palmitoleic, oleic, elaidic, asclepinic, gondoleic, eicosenoic, sorbic, linoleic, linolenic, punicic, stearidonic, arachidonic and stearolic radicals, and mixtures thereof.

Preferably, at least one dextrin palmitate is used as dextrin ester of fatty acid(s). This ester may be used alone or as a mixture with other esters.

Advantageously, the dextrin ester of fatty acid has a degree of substitution of less than or equal to 2.5, especially ranging from 1.5 to 2.5, and preferably from 2 to 2.5, on the basis of one glucose unit. The weight-average molecular weight of the dextrin ester may in particular be from 10 000 to 150 000, especially from 12 000 to 100 000, or even from 15 000 to 80 000.

Dextrin esters, in particular dextrin palmitates, are commercially available under the name Rheopearl TL or Rheopearl KL by the company Chiba Flour.

Diblock, triblock, multiblock, radial-block or star copolymers

The block polymers that are suitable for use in the invention are especially those described in patents U.S. Pat. No. 5,756,082 and EP 0 497 144, and also in patent application WO 98/42298.

Also, the block polymers that may be used in the present invention may be chosen from:

block (diblock or triblock) copolymers such as the polystyrene silicones or the polyethylene silicones described in patents U.S. Pat. No. 6,225,390, U.S. Pat. No. 6,160,054, U.S. Pat. No. 6,174,968 and U.S. Pat. No. 6,225,390,

block or grafted copolymers comprising a silicone block and another block or graft of polyvinyl or polymethacrylic type, such as those described in patents U.S. Pat. No. 5,468,477 and U.S. Pat. No. 5,725,882,

polymers or copolymers resulting from the polymerization or copolymerization of an ethylenic monomer, comprising one or more optionally conjugated ethylenic bonds (or dienes),

polymers or copolymers resulting from the polymerization or copolymerization of an ethylenic monomer, especially a copolymer of vinyl, acrylic or methacrylic type, which may be a block copolymer, such as a diblock, triblock or even multiblock copolymer or a radial or star copolymer.

The gelling agent of ethylenic type may comprise, for example, a styrene block, an alkylstyrene block, an ethylene/butylene block, an ethylene/propylene block, a butadiene block, and isoprene block, an acrylate block or a methacrylate block, or a combination of these blocks.

According to one embodiment, the diblock, triblock, multiblock and/or radial or star copolymers may comprise at least two thermodynamically incompatible segments.

A diblock copolymer is usually defined as being of A-B type or as a block in which a hard segment (A) is followed by a soft segment (B).

A triblock copolymer is usually defined as being of A-B-A type or as a ratio of a hard segment, a soft segment and a hard segment.

A multiblock, radial or star copolymer may comprise any type of combination of hard segments and soft segments, with the proviso that the characteristics of the hard segments and of the soft segments are conserved.

An example of hard segments of block copolymers that may be mentioned is styrene, and examples of soft segments of block copolymers that may be mentioned include ethylene, propylene and butylene, and a combination thereof.

The triblock copolymers, and especially those of polystyrene/polyisoprene or polystyrene/polybutadiene type, which is suitable for use in the invention may be those sold under the reference Luvitol HSB by the company BASF. Mention may also be made of triblock copolymers of polystyrene/copoly(ethylene-propylene) or polystyrene/copoly(ethylene-butylene) type, such as those sold under the reference Kraton by the company Shell Chemical Co., or under the reference Gelled Permethyl 99 A by the company Penreco.

As a further example of block copolymers that may be suitable for use in the present invention, mention may also be made of the block copolymers sold under the reference Versagel by the company Penreco, those sold under the reference Kraton by the company Shell and those sold under the reference Gel Base by the company Brooks Industries.

Among the oily-phase-gelling polymers that are suitable for use in the invention, mention may be made especially of the copolymer of ethylenediamine/stearyl dimerdilinoleate and the semi-crystalline polymer of poly-C₁₀₋₃₀ alkyl acrylate type, and mixtures thereof.

According to one embodiment variant of the present invention, the structured calibrated oily particles may especially contain isopropyl lauroylsarcosinate as oil, and an ethylenediamine dimerdilinoleate/stearyl copolymer as gelling polymer.

According to another embodiment variant, the structured calibrated oily particles may comprise squalane as oil and a semi-crystalline polymer of poly-C₁₀₋₃₀ alkylacrylate type as gelling polymer.

According to yet another embodiment, the structured calibrated oily particles may comprise a mixture of isopropyl laurylsarcosinate and isononyl isononanoate as oily phase and the ethylenediamine dimerdilinoleate/stearyl copolymer as gelling polymer.

According to yet another embodiment, the structured calibrated oily particles in accordance with the present invention may comprise a mixture of isononyl isononanoate and octyldodecanol as oily phase and the ethylenediamine dimerdilinoleate/stearyl copolymer as gelling polymer.

In addition, the particles may comprise another gelling agent of alkylglutamic acid derivative type, for example the laurylglutamic acid dibutylamide sold by the company Ajinomoto under the name “Gelling Agent GP-1”.

Active Substances

The particles according to the invention may also comprise at least one active substance.

The active substances incorporated into the particles in accordance with the invention are advantageously lipophilic. They may be chosen especially from substances suitable for making up and/or caring for the skin, including, for example, the scalp and the lips.

In the context of the present invention, the terms “active agent” and “active substance” mean a compound which by itself, i.e. not requiring the intervention of an external agent to activate it, has biological activity.

The biological activity of the active agent may be useful in particular for caring for greasy skin, which may be in particular:
desquamating activity, and/or
antimicrobial activity (especially on P. acnes), and/or
anti-inflammatory activity, and/or
sebo-regulatory activity, and/or
antioxidant activity.

In this respect, the active agent may thus be chosen from: desquamating agents and/or antimicrobial agents and/or anti-inflammatory agents and/or sebo-regulatory agents and/or antioxidants.

1. Desquamating Agents

The term “desquamating agent” means any compound capable of acting:
either directly on desquamation by promoting exfoliation, such as β-hydroxy acids, in particular salicylic acid and its derivatives (including 5-n-octanoylsalicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol and certain jasmonic acid derivatives;
or on the enzymes involved in the desquamation or degradation of corneodesmosomes, glycosidases, stratum corneum chymotryptic enzyme (SCCE), or even other proteases (trypsin, chymotrypsin-like). Mention may be made of aminosulfonic compounds and in particular (N-2-hydroxyethylpiperazine-N-2-ethane)sulfonic acid (HEPES); 2-oxothiazolidine-4-carboxylic acid (procysteine) derivatives; α-amino acid derivatives of the type glycine (as described in EP-0 852 949 and sodium methylglycinediacetate sold by BASF under the trade name Trilon M); honey; sugar derivatives such as O-octanoyl-6-D-maltose and N-acetylglucosamine.

5-n-Octanoylsalicylic acid is preferred for use in the present invention.

2. Antimicrobial Agents

The antimicrobial agents that may be used in the composition according to the invention may be chosen especially from 2,4,4′-trichloro-2′-hydroxydiphenyl ether (or triclosan), 3,4,4′-trichlorobanilide, phenoxyethanol, phenoxypropanol, phenoxyisopropanol, hexamidine isethionate, metronidazole and its salts, micronazole and its salts, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, 2,4,4′-trichloro-2′-hydroxydiphenyl ether, 3,4,4′-trichlorocarbanalide, octopirox, octoxyglycerine, octanoylglycine, caprylyl glycol, 10-hydroxy-2-decanoic acid, dichlorophenylimidazole dioxolane and its derivatives described in patent WO 93/18743, copper pidolate, salicylic acid, iodopropynyl butyl carbonate, famesol and phytosphingosines, and mixtures thereof.

The preferred antimicrobial agents are octoxyglycerol, copper pidolate, salicylic acid and iodopropynyl butyl carbamate.

3. Anti-Inflammatory Agents

As anti-inflammatory agents or calmatives that may be used in the composition according to the invention, mention may be made of: pentacyclic triterpenes and extracts of plants (e.g.: Glycyrrhiza glabra) containing them, for instance α-glycyrrhetinic acid and salts and/or derivatives thereof (glycyrrhetinic acid monoglucoronide, stearyl glycyrrhetinate or 3-stearoyloxyglycyrrhetic acid), ursolic acid and its salts, oleanolic acid and its salts, betulinic acid and its salts, an extract of Paeonia suffruticosa and/or lactiflora, salicylic acid salts and in particular zinc salicylate, the phycosaccharides from the company Codif, an extract of Laminaria saccharina, canola oil, bisabolol and camomile extracts, allantoin, Sepivital EPC (phosphoric diester of vitamins E and C) from SEPPIC, omega-3 unsaturated oils such as musk rose oil, blackcurrant oil, ecchium oil, fish oil, plankton extracts, capryloylglycine, Seppicalm VG (sodium palmitoylproline and Nymphea alba) from SEPPIC, an extract of Pygeum, an extract of Boswellia serrata, an extract of Centipeda cunnighami, an extract of Helianthus annuus, an extract of Linum usitatissimum, tocotrienols, extracts of Cola nitida, extracts of Centella asiatica, piperonal, an extract of clove, an extract of Epilobium angustifolium, Aloe vera, an extract of Bacopa monieri, phytosterols, niacinamide, cortisone, hydrocortisone, indomethacin and betamethasone.

The preferred anti-inflammatory agents for use in the present invention are extracts of Centella asiatica, β-glycyrrhetinic acid and its salts, α-bisabolol and niacinamide.

4. Sebo-Regulatory Agents

When the composition according to the invention comprises a sebo-regulatory agent such as a 5α-reductase inhibitor, this agent may be chosen especially from:
retinoids, and in particular retinol;
sulfur and sulfur derivatives;
zinc salts such as zinc lactate, gluconate, pidolate, carboxylate, salicylate and/or cysteate;
selenium chloride;
vitamin B6 or pyridoxine;
mixture of capryloyl glycine, sarcosine and extract of Cinnamomum zeylanicum sold especially by the company SEPPIC under the trade name Sepicontrol A5®;
an extract of Laminaria saccharina sold especially by the company SECMA under the trade name Phlorogine®;
an extract of Spiraea ulmaria sold especially by the company Silab under the trade name Sebonormine®;
plant extracts from the species Arnica montana, Cinchona succirubra, Eugenia caryophyllata, Humulus lupulus, Hypericum perforatum, Mentha piperita, Rosmarinus officinalis, Salvia oficinalis and Thymus vulgaris, all sold, for example, by the company Maruzen;
an extract of Serenoa repens sold especially by the company Euromed;
plant extracts of the genus Silybum;
plant extracts containing sapogenins and in particular extracts of diosgenin-rich Dioscorea plants; and
extracts of Eugenia caryophyllata containing eugenol or eugenyl glucoside.

Zinc salts are preferred for use in the present invention.

Antioxidants

The antioxidants that are preferred for use in the present invention may be chosen from tocopherol and esters thereof, such as tocopheryl acetate; BHT and BHA.

All these active agents are particularly useful as active agents targeting the sebaceous follicles.

Other active agents may also be used in the context of the present invention, as active agents more specifically targeting the hair follicles.

Examples of these active agents that may be mentioned include:
agents for inhibiting hair loss and also agents for stimulating hair growth, such as minoxidil, biotin, finasteride, 2,4-dipyrimidine N-oxide, panthenol and derivatives thereof, flavanone T, or, more generally, any plant extract, having activity against type I or II 5-α-reductase;
agents for inhibiting the growth of head hair or body hairs, such as the serine proteases described in patent U.S. Pat. No. 6,407,056, caffeic acid, quercetin, propyl gallate, norhydroguaiaretic acid or NDGA, indomethacin, eflornithine hydrochloride, plant extracts as described in patent U.S. Pat. No. 6,171,595, for instance extracts of clove, of rosebuds, of burnet or of Bengal Gambier, the compounds described in patent U.S. Pat. No. 6,075,052, tetramisole, sodium orthovandate, levamisole, disodium chromoglycate, vanadium nitrate and gallium nitrate as described in patent U.S. Pat. No. 6,020,006, and also the compounds described in patents U.S. Pat. No. 4,885,289, U.S. Pat. No. 4,720,489, U.S. Pat. No. 5,132,293, U.S. Pat. No. 5,096,911, U.S. Pat. No. 5,095,007, U.S. Pat. No. 5,143,925, U.S. Pat. No. 5,328,686, U.S. Pat. No. 5,440,090, U.S. Pat. No. 5,364,885, U.S. Pat. No. 5,411,991, U.S. Pat. No. 5,648,394, U.S. Pat. No. 5,468,476, U.S. Pat. No. 5,475,763, U.S. Pat. No. 5,455,608, U.S. Pat. No. 5,674,477, U.S. Pat. Nos. 5,728,736 and 5 652 273 and in patent applications WO 94/27586, WO 94/27563 and WO 98/03149. Extracts of juniper may also be used, as described in patent U.S. Pat. No. 6,375,948;
antidandruff agents such as zinc pyrithione; and
mixtures thereof.

As examples of active substances more particularly suitable for use in the invention, mention may be made of 5-n-octanoylsalicyclic acid, α-bisabolol, α-tocopherol, iodopropynyl butyl carbamate and ursolic acid, and mixtures thereof.

The content of active substance(s) or molecule(s) present in the particles in accordance with the invention may range from 0.05% to 70% by weight, from 0.1% to 50% by weight and in particular from 0.5% to 40% by weight relative to the total weight of the particle.

Surfactants

The particles according to the invention may also comprise at least one surfactant.

The presence of (a) surfactant(s) and the chemical nature thereof are generally determined by the nature of the process for preparing the said particles.

Thus, when the particles are prepared according to the process described below, involving an emulsification step, at least one surfactant chosen from nonionic surfactants and ionic surfactants, and mixtures thereof, is introduced into the oily phase-aqueous phase mixture.

The nonionic surfactants, or mixtures thereof, advantageously used in the context of the present invention are surfactants, or mixtures thereof, with an HLB of greater than 5.

As examples of nonionic surfactants that are suitable for use in the invention, mention may be made of:

oxyethylenated or non-oxyethylenated monoalkyl or polyalkyl esters or ethers of glycerol, such as those described in patent U.S. Pat. No. 6,541,018;

oxyethylenated or non-oxyethylenated monoalkyl or polyalkyl esters or ethers of sorbitan, such as those described in U.S. Pat. No. 6,335,022;

monoalkyl or polyalkyl esters or ethers of polyethylene oxide, such as those described in U.S. Pat. No. 6,375,960;

oxyethylenated or non-oxyethylenated monoalkyl or polyalkyl esters or ethers of sugars, such as those described in U.S. Pat. No. 6,689,371; and

mixtures thereof.

The ionic surfactants that may be used in the context of the present invention may be of anionic type, of cationic type or of amphiphilic type.

The anionic surfactants may be chosen especially from:

alkoxylated alkenylsuccinates such as those mentioned in patent U.S. Pat. No. 6,461,625;

alkyl ether citrates such as those mentioned in patent U.S. Pat. No. 6,413,527;

phosphoric alkyl esters such as those described in patent U.S. Pat. No. 6,274,150; and

mixtures thereof.

The alkyl chains of the anionic surfactants that are suitable for use in the invention are advantageously included in the range from C₁₂ to C₂₄, and may be saturated or unsaturated and/or linear or branched.

The ionic surfactants that may be used for the present invention may also be lipoamino acids or alkylsulfonic derivatives, and mixtures thereof.

The lipoamino acids may be chosen especially from monosodium and disodium acylglutamates, for instance the disodium salt of N-stearoyl-L-glutamic acid sold under the name Acylglutamate HS21 by the company Ajinomoto.

The alkylsulfonic derivatives may be chosen especially from the alkylsulfonic derivatives of the first formula below: 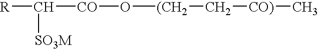
in which R represents an alkyl radical containing from 16 to 22 carbon atoms, and especially a C₁₆H₃₃ or C₁₈H₃₇ radical, taken as a mixture or separately, and M is an alkali metal, for instance sodium.

The cationic surfactants that are suitable for preparing the particles and/or dispersions in accordance with the invention may be chosen especially from quaternary ammonium salts and fatty amines and salts thereof, and mixtures thereof.

The quaternary ammonium salts are, for example:

a) those having the second general formula below: 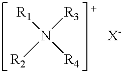
in which the radicals R₁ to R₄, which may be identical or different, represent a linear or branched aliphatic radical containing from 1 to 30 carbon atoms or an aromatic radical such as aryl or alkylaryl.

The aliphatic radicals may comprise heteroatoms especially such as oxygen, nitrogen, sulfur and halogens. The aliphatic radicals are chosen, for example, from alkyl, alkoxy, polyoxy(C₂-C₆)alkylene, alkylamide, (C₁₂-C₂₂)alkylamido(C₂-C₆)alkyl, (C₁₂-C₂₂)alkylacetate and hydroxyalkyl, containing from about 1 to 30 carbon atoms; X is an anion chosen from the group of halides, phosphates, acetates, lactates, (C₂-C₆)alkyl sulfates, and alkyl- or alkylarylsulfonates.

As quaternary ammonium salts of the second formula presented above, which are advantageously used, mention may be made, firstly, of tetraalkylammonium chlorides, for instance dialkyldimethylammonium or alkyltrimethylammonium chlorides, in which the alkyl radical contains from about 12 to 22 carbon atoms, in particular behenyltrimethylammonium, distearyldimethylammonium, cetyltrimethylammonium or benzyldimethylstearylammonium chloride, or alternatively, or, secondly, of stearamidopropyldimethyl(myristyl acetate)ammonium chloride sold under the name “Ceraphyl 70” by the company Van Dyk.

b) the quaternary ammonium salts of imidazolinium, for instance those of the third general formula below: 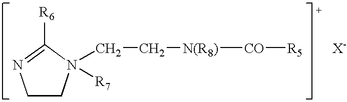
in which R₅ represents an alkenyl or alkyl radical containing from 8 to 30 carbon atoms, for example fatty acid derivatives of tallow; R₆ represents a hydrogen atom, an alkyl radical containing from 1 to 4 carbon atoms or an alkenyl or alkyl radical containing from 8 to 30 carbon atoms; R₇ represents an alkyl radical containing from 1 to 4 carbon atoms; R₈ represents a hydrogen atom or an alkyl radical containing from 1 to 4 carbon atoms; X is an anion chosen from the group of halides, phosphates, acetates, lactates, alkyl sulfates, and alkyl- or alkylarylsulfonates.

In particular, R₅ and R₆ denote a mixture of alkenyl or alkyl radicals containing from 12 to 21 carbon atoms, for example fatty acid derivatives of tallow, R₇ denotes a methyl radical, R₈ denotes hydrogen. Such a product is sold, for example, under the name “Rewoquat W 75” by the company Rewo.

c) the diquaternary ammonium salts of the fourth general formula below: 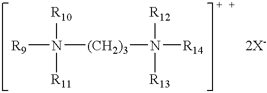
in which R₉ denotes an aliphatic radical containing from about 16 to 30 carbon atoms; R₁₀, R₁₁, R₁₂, R₁₃ and R₁₄ are chosen from hydrogen and an alkyl radical containing from 1 to 4 carbon atoms; and X is an anion chosen from the group of halides, acetates, phosphates, nitrates and methyl sulfates. Such diquaternary ammonium salts especially include propane tallow diammonium dichloride.

As examples of surfactants that are suitable for use in the invention, mention may be made of PEG-30 glyceryl stearate and disodium stearoylglutamate, and mixtures thereof.

According to yet another embodiment variant, the dispersions in accordance with the present invention may comprise as nonionic surfactant a mixture of PEG-30 glyceryl stearate and of disodium stearoylglutamate.

The content of nonionic surfactant and/or ionic surfactant used for the preparation of the particles and/or dispersions in accordance with the invention may advantageously range from 0.5% to 50% by weight, or even from 1% to 40% by weight and in particular from 5% to 20% by weight relative to the total weight of the dispersion.

Emulsifying Polymers

The particles may also contain an emulsifying polymer, i.e. an amphiphilic polymer.

Among the emulsifying polymers that are suitable for use in the invention, mention may be made of:

POE-POP diblock and triblock copolymers such as those described in patent U.S. Pat. No. 6,464,990;

polyoxyethylenated silicone surfactants such as those described in patent U.S. Pat. No. 6,120,778;

non-crosslinked hydrophobic AMPSs such as those described in EP 1 466 588;

amphiphilic acrylic polymers, such as PEMULEN TR-1 or TR-2 or equivalent;

the associative and gelling polymers described in US 2003/0138465;

heat-gelling polymers such as those described in patent applications US 2004/0214913, US 2003/0147832 and US 2002/0198328 and FR2 856 923.

When they are present, the emulsifying polymer(s) may be introduced in a content ranging from 0.1% to 15% by weight, or even from 0.1% to 10% by weight and more particularly from 0.1% to 5% by weight relative to the total weight of the dispersion.

Process for Obtaining the Dispersions

The structured, calibrated and spherical oily particles in accordance with the invention may be obtained in the form of a dispersion by means of a process comprising at least the steps consisting in:

emulsifying a mixture of at least one oil or an oily phase and at least one oily-phase-gelling polymer with an aqueous and/or water-soluble phase at a temperature above the gel point of the polymer,

subjecting the mixture to a process leading to the production of oily particles, at a temperature at least 5 to 10° C. above the melting point of the mixture used in the preceding step, and

cooling the particle dispersion thus obtained.

It is pointed out that the presence of water in the first step of the process and the execution of the second step with heating are cumulative conditions necessary for obtaining spherical calibrated particles according to the invention.

The viscosity measurement is indeed carried out on the initial mixture, i.e. on the “macrogel” rather than when the particles are already formed, as has already been pointed out hereinabove.

The process according to the invention may, where appropriate, also include a step consisting in diluting the continuous phase of the mixture before the cooling step.

For the purposes of the present invention, the expression “process leading to the production of oily particles” is intended to denote an action of shear type or a mechanism of phase inversion type.

The temperature at which the emulsification step is performed is advantageously greater than 40° C. and advantageously less than 95° C.

Thus, after the process, the dispersions in accordance with the invention comprise in an aqueous and/or water-soluble phase calibrated oily particles comprising an oily phase structured with at least one gelling polymer.

The nature of the process exerted on the oily phase/gelling polymer mixture determines the size of the particles to be obtained.

Thus, for submicron particles, with a mean size of about from 150 nm to 1 μm, it is advantageously possible to use processes that develop a turbulent shear, such as ultrasonication, high-pressure homogenization (working pressure of between 50 and 1000 bar), for example using a Soavi OBL 20® machine from Niro Soavi, or the Microfluidizer® machine from Microfluidics. Processes not requiring any input of mechanical energy may also be used, such as those involving a phase inversion during the emulsification, for instance PIT (phase inversion temperature) or composition inversion (for example by adding a hydrophilic surfactant to a W/O emulsion to invert it to an O/W emulsion), and nanoprecipitation as described in EP 0 447 318.

For the micron-sized particles, with a mean size of about from 1 μm to 15 μm, it is possible to use, for example, processes for obtaining the smallest possible polydispersity, such as the controlled shear of viscoelastic emulsions, as described in patent application FR2 747 321 and patent U.S. Pat. No. 5,558,820, continuous processes as described in patent U.S. Pat. No. 5,688,842 and patent application WO 02/40574, or those more generally using a colloidal mill, a static mixer, a micromixer, a frame paddle or alternatively a porous membrane, as described in patent U.S. Pat. No. 5,326,484. It is also possible to use processes involving maturation control (U.S. Pat. No. 6,160,061), the swelling of a “templating agent” latex (EP 719 087), Rayleigh instabilities (Weitz, Langmuir, 16, 347-351, (2000)) or fractionation of polydisperse emulsions (Bibette, J. Coll. Int. Sci., vol 147, No. 2, 474-478, (1991)).

In order to facilitate the formation of the particles, during the emulsification step, it is possible, for example, to use one or more nonionic or ionic surfactants and/or hydrophobic emulsifying polymers, as defined above.

Moreover, in the case, for example, of processes developing a laminar shear in order to obtain a uniform particle size distribution, it may optionally be advantageous to adjust the ratio between the viscosity of the dispersed oily phase and the viscosity of the continuous aqueous and/or hydrophilic phase in a ratio ranging from 0.01 to 5 or even from 0.05 to 2. This adjustment may especially be performed by adding surfactants and/or emulsifying polymers such as those described above and/or hydrophilic gelling polymers.

Thus, according to one embodiment of the invention, the aqueous and/or water-soluble continuous phase may also comprise at least one gelling hydrophilic polymer.

Advantageously, when it is present, the gelling hydrophilic polymer is introduced into the aqueous, or hydrophilic, continuous phase in a proportion ranging from 0.1% to 30% by weight and especially from 0.05% to 15% by weight relative to the total weight of the composition.

As examples of gelling hydrophilic polymers, mention may be made especially of carbomers, acrylamidomethylpropanesulfonic (AMPS) derivatives, cellulose derivatives or guar derivatives. As guar derivatives that may advantageously be used in the implementation of the present invention, mention may be made of the hydroxypropyl guar sold under the reference Jaguar HP® 105 by the company Rhodia.

Thus, the oily phase-aqueous and/or water-soluble phase mixture may also comprise a compound chosen from a surfactant, an emulsifying polymer, a hydrophilic gelling polymer, and mixtures thereof, and preferably a mixture of a surfactant, an emulsifying polymer and a hydrophilic gelling polymer.

According to one variant, the oily phase-aqueous phase mixture may also comprise at least one active substance. This active substance may be as defined above.

Thus, on account of the process for preparing the particles in accordance with the invention, these particles are advantageously free of volatile solvent.

Dispersion

In accordance with the process for obtaining the particles in accordance with the invention, as described above, these particles are obtained as a dispersion in a continuous and/or water-soluble phase.

The content of calibrated oily particles, comprising an oily phase structured with a gelling polymer, according to the invention, present in the aqueous and/or water-soluble continuous phase may especially be such that the oily mass fraction dispersed in the aqueous and/or water-soluble phase may range from 5% to 89% by weight, especially from 20% to 85% by weight or even from 40% to 80% by weight and in particular from 60% to 80% by weight relative to the total weight of the dispersion.

The structured calibrated oily particles in accordance with the invention advantageously do not aggregate in the dispersion in which they are obtained, and their granulometric specificities in terms of size and distribution index are advantageously conserved therein.

The aqueous and/or water-soluble continuous phase that is suitable for use in the invention may advantageously be water and/or a water-soluble organic solvent, for instance glycols such as glycerol or dipropylene glycol, alone or as mixtures.

For the purposes of the present invention, the term “water-soluble solvent” is intended to denote a compound that is liquid at room temperature and water-miscible (miscibility in water of greater than 50% by weight at 25° C. and at atmospheric pressure).

Among the water-soluble solvents that may be used in the dispersions in accordance with the invention, mention may be made especially of lower monoalcohols containing from 1 to 5 carbon atoms, such as ethanol and isopropanol, glycols containing from 2 to 8 carbon atoms, such as ethylene glycol, propylene glycol, 1,3-butylene glycol and dipropylene glycol, C₃ and C₄ ketones, glycerol and C₂-C₄ aldehydes.

According to yet another embodiment variant, the dispersions in accordance with the present invention may comprise demineralized water as continuous aqueous phase.

According to yet another embodiment variant, the dispersions in accordance with the invention may comprise as nonionic surfactant a mixture of PEG-30 glyceryl stearate and disodium stearoylglutamate, and hydroxypropyl guar as hydrophilic gelling polymer.

Cosmetic or Dermatological Composition

The particles and/or dispersions in accordance with the invention, comprising an active substance, may allow the specific administration of cosmetically and/or dermatologically active substances into the sebaceous glands and/or the hair follicles. These particles and/or dispersions may advantageously be introduced into various cosmetic and/or dermatological formulations intended for topical application to the skin and the hair.

Thus, the particles and/or dispersions in accordance with the present invention may be used as vehicles for at least one active substance for the preparation of (a) cosmetic and/or dermatological composition(s) that may be used in the field of caring for and/or making up the skin, to promote the at least partial penetration of the active substance into the sebaceous glands and/or the hair follicles.

Thus, a subject of the present invention is also cosmetic or dermatological compositions comprising at least some particles and/or at least one dispersion as defined above.

The compositions comprising particles and/or dispersions in accordance with the invention may be care, hygiene and/or makeup compositions, for the skin or the integuments.

In the present case, a composition according to the invention may be in the form of a mascara, an eyebrow product, an eyeliner, an eyeshadow, a makeup rouge, a foundation, a lip product, a body makeup product, a hair makeup product or a haircare product.

They may be intended, for example, for haircare use and may be in particular shampoos, hair conditioners or hair lotions, in particular haircare lotions.

Advantageously, the composition may contain from 0.01% to 40% by weight, especially from 0.1% to 25% by weight or even from 0.2% to 20% by weight of particles in accordance with the present invention relative to the total weight of the composition.

The cosmetic or dermatological composition may be in the form of a lotion, an oil-in-water (O/W) or water-in-oil (W/O) or multiple (W/O/W) emulsion, an aqueous or aqueous-alcoholic gel, a cream, a milk, etc.

Additives

The cosmetic compositions in accordance with the invention may also comprise any additive usually used in the field under consideration, with the proviso that these additives do not impair the property of penetration of the compositions into the sebaceous glands and/or the hair follicles.

In the context of compositions intended especially for makeup, the additives that may be suitable for use in the invention may be chosen especially from dyestuffs, for instance nacres and pigments, fillers, antioxidants, film-forming agents and, where appropriate, film-forming auxiliaries, essential oils, preserving agents, fragrances, moisturizers, antiseptics and neutralizers, and mixtures thereof.

Needless to say, a person skilled in the art will also take care to select the possible additional additives and/or the amount thereof such that the advantageous properties of the composition according to the invention are not, or are not substantially, adversely affected by the intended addition.

A subject of the present invention is also a non-therapeutic cosmetic makeup and/or care process, comprising at least the step of applying a composition as defined above to the skin.

The examples of dispersions of particles and of compositions presented hereinbelow are given as illustrations and with no limiting nature on the invention.

### EXAMPLES

### Example 1

### Microparticles Containing a Keratolytic Agent

The dispersion is prepared in a beaker with an inside diameter of 72 mm comprising a jacket to allow its temperature to be regulated by means of a heating bath. An aqueous solution composed of 12.8 g of PEG-30 glyceryl stearate (TagatS® from the company Degussa), 3.2 g of disodium stearoylglutamate (Amisoft HS21P® from the company Ajinomoto) and 64 g of demineralized water is introduced therein. The mixture is brought to 85° C.

The following mixture is separately prepared: 30 g of ethylenediamine dimerdilinoleate/stearyl copolymer (Uniclear 100 VG® from the company Arizona Chemical), 84 g of isopropyl lauroylsarcosinate (Eldew SL-205® from the company Ajinomoto) and 6 g of 5-n-octanoylsalicyclic acid, which is brought to a temperature of 85° C.

Once the mixture is homogeneous, it is introduced over 10 minutes into the aqueous surfactant solution while stirring with a paddle 70 mm wide and 2 mm thick at a spin speed of 160 rpm. After 3 minutes, the spin speed is raised to 300 rpm and maintained for 30 min.

The emulsion is then diluted with 100 g of demineralized water at 80° C. to achieve a mass fraction of dispersed phase of 40%, and is then cooled to room temperature.

A stable, homogeneous dispersion of microparticles with a granulometry of 2.47 μm (d[0.5]) and a uniformity coefficient (U) of 0.27 is obtained.

### Example 2

### Microparticles Containing α-Bisabolol

The dispersion is prepared in a beaker with an inside diameter of 72 mm comprising a jacket to allow its temperature to be regulated by means of a heating bath. An aqueous solution composed of 6.4 g of PEG-30 glyceryl stearate (Tagat S® from the company Degussa), 1.6 g of disodium stearoylglutamate (Amisoft HS21P® from the company Ajinomoto) and 32 g of demineralized water is introduced therein. The mixture is brought to 80° C.

The following mixture is separately prepared: 48 g of poly(C10-30 alkyl acrylate) (Doresco IPA 13-6® from the company Landec Corporation), 56 g of squalane and 56 g of α-bisabolol, which is brought to a temperature of 80° C.

Once the mixture is homogeneous, it is introduced over 10 minutes into the aqueous surfactant solution while stirring with a paddle 70 mm wide and 2 mm thick with a spin speed of 160 rpm. After 3 minutes, the spin speed is raised to 400 rpm and maintained for 30 minutes.

The emulsion is then diluted with 200 g of demineralized water at 80° C. to achieve a mass fraction of dispersed phase of 40%, and is then cooled to room temperature.

A homogeneous dispersion of microparticles with a granulometry of 4.2 μm (d[0.5]) and a uniformity coefficient (U) of 0.25 is obtained.

### Example 3

### Microparticles Containing Vitamin E and an Antibacterial Agent

The dispersion is prepared in a beaker with an inside diameter of 72 mm comprising a jacket to allow its temperature to be regulated by means of a heating bath. An aqueous solution composed of 1.92 g of PEG-30 glyceryl stearate (Tagat S® from the company Degussa), 0.48 g of disodium stearoylglutamate (Amisoft HS21P® from the company Ajinomoto), 1.2 g of hydroxypropyl guar (Jaguar HP105 from the company Rhodia) and 116.4 g of demineralized water is introduced therein. The mixture is brought to 80° C.

The following mixture is separately prepared: 20 g of ethylenediamine dimerdilinoleate/stearyl copolymer (Uniclear 100 VG® from the company Arizona Chemical), 28.24 g of isopropyl lauroylsarcosinate (Eldew SL-205® from the company Ajinomoto), 28.24 g of isononyl isononanoate, 3.2 g of α-tocopherol and 0.32 g of iodopropynyl butyl carbamate (Glycacil® from the company Lonza), which is brought to a temperature of 80° C.

Once the mixture is homogeneous, it is introduced over 10 minutes into the aqueous surfactant solution while stirring with a paddle 70 mm wide and 2 mm thick with a spin speed of 160 rpm. After 3 minutes, the spin speed is raised to 400 rpm and maintained for 30 minutes.

The emulsion is then diluted with 150 g of demineralized water at 80° C. to achieve a mass fraction of dispersed phase of 23%, and is then cooled to room temperature.

A homogeneous dispersion of microparticles with a granulometry of 7.18 μm (d[0.5]) and a coefficient of uniformity (U) of 0.40 is obtained.

### Example 4

### Ursolic Acid Nanoparticles

An aqueous solution composed of 9 g of PEG-30 glyceryl stearate, 1 g of disodium stearoylglutamate and 390 g of demineralized water is prepared. The mixture is brought to 85° C. Separately, the following mixture is prepared: 30 g of ethylenediamine dimerdilinoleate/stearyl copolymer (Uniclear 100 VG® from the company Arizona Chemical), 59.5 g of isononyl isononanoate, 10 g of octyldodecanol and 0.5 g of ursolic acid, and this mixture is brought to a temperature of 85° C.

It is introduced into the aqueous surfactant solution while stirring with a device of rotor-stator type (Ultra-Turrax T50). After 5 minutes, the mixture is homogenized at 85° C. using a Soavi Panda homogenizer, by homogenization twice at a pressure of about 400 bar.

The dispersion is then cooled to room temperature.

A homogeneous dispersion of nanoparticles with a mean diameter of 250 nm is obtained.

### Example 5

### O/W Emulsion Containing the Microparticles of the Invention

| Phase A | |
|---|---|
| Disodium EDTA | 0.10% |
| Glycerol | 5.00% |
| Preserving agent | 0.20% |
| Demineralized water | 50.00% |
| Phase B | |
| Polyglyceryl-3 methylglucose distearate (Tego Care 450 | 1.50% |
| from the company Degussa) | |
| Glyceryl stearate SE (Tegin Vegetable Based from the | 1.50% |
| company Degussa) | |
| Stearyl alcohol | 1.00% |
| Stearic acid | 2.00% |
| Vitis vinifera oil from grapeseed | 1.00% |
| Prunus armeniaca oil from apricot kernel | 2.00% |
| Preserving agent | 0.20% |
| Phase C | |
| Cyclohexasiloxane | 2.00% |
| Dimethicone (and) dimethiconol (DC 2-9085 from | 1.00% |
| the company Dow Corning) | |
| Phase D | |
| Carbomer | 0.40% |
| Demineralized water | 15.00% |
| Phase E | |
| Xanthan gum | 0.20% |
| Demineralized water | 7.00% |
| Phase F | |
| Triethanolamine | 0.40% |
| Demineralized water | 2.00% |
| Phase G | |
| Dispersion of microparticles of Example 1 | 7.50% |

A fine and light emulsion for combating greasy skin is obtained.

### Example 6

### O/W Emulsion Containing the Microparticles of the Invention

| Phase A | |
|---|---|
| PEG-20 methylglucose sesquistearate (GLUCAMATE | 2.00% |
| SSE 20 from Chemron) | |
| Disodium EDTA | 0.10% |
| Glycerol | 5.00% |
| Preserving agent | 0.20% |
| Demineralized water | 44.70% |
| Phase B | |
| Methylglucose sesquistearate | 2.00% |
| Stearyl alcohol (and) Ceteareth-20 | 2.00% |
| Cyclohexasiloxane | 5.85% |
| C12-15 alkyl benzoate | 5.85% |
| Preserving agent | 0.10% |
| Phase C | |
| Polyacrylamide (and) C13-14 isoparaffin (and) | 1.00% |
| Laureth-7 (Sepigel 305 from SEPPIC) | |
| Ammonium polyacryloyldimethyl taurate (Hostacerin | 1.20% |
| AMPS from Clariant) | |
| Phase D | |
| Biosaccharide Gum-1 (Fucogel 1000PP from Solabia) | 5.00% |
| Phase E | |
| Aluminium starch octenylsuccinate | 3.00% |
| Phase F | |
| Microparticle dispersion of Example 3 | 22.00% |

A soft and fresh cream suitable for treating acne is obtained.

### Example 7

### Microparticles Containing β-Glycyrrhetinic Acid

The dispersion is prepared in a beaker with an inside diameter of 72 mm comprising a jacket for regulating its temperature by means of a heating bath. An aqueous solution composed of 6.4 g of PEG-30 glyceryl stearate (Tagat S® from the company Degussa), 1.6 g of disodium stearoylglutamate (Amisoft HS21P® from the company Ajinomoto) and 32 g of demineralized water is introduced therein. The mixture is brought to 85° C.

The following mixture is separately prepared:
40 g of Nylon-611/dimethicone copolymer (DC2-8178 Gellant from the company Dow Corning),
60 g of dimethicone (DC Fluid 200-5 cs from the company Dow Corning),
57 g of isopropyl lauroylsarcosinate (Eldew SL-205 from the company Ajinomoto), and
3 g of β-glycyrrhetinic acid, which is brought to a temperature of 85° C.

Once the mixture is homogeneous, it is introduced over 10 minutes into the aqueous surfactant solution while stirring with a paddle 70 mm wide and 2 mm thick, at a spin speed of 160 rpm. After 3 minutes, the spin speed is raised to 400 rpm and maintained for 30 minutes.

The emulsion is then diluted with 200 g of demineralized water at 80° C. to achieve a mass fraction of dispersed phase of 40%, and is then cooled to room temperature.

A homogeneous dispersion of microparticles with a granulometry of 1.2 μm (d[0.5]) and a coefficient of uniformity (U) of 0.34 is obtained.

What is claimed is:

1. Calibrated and spherical oily particles, comprising at least one oily phase structured with at least one gelling polymer, wherein:
the particles have a mean size of less than or equal to 15 μm;
the structured oily phase has a melting point of greater than or equal to 40° C.; and
the particles have a circularity index of from 0.9 to 1.

2. The particles of claim 1, wherein the particles have a mean size of less than or equal to 11 μm.

3. The particles of claim 1, wherein the particles have a mean size of from 150 nm to 11 μm.

4. The particles of claim 1, wherein the structured oily phase has a melting point of from 50° C. to 90° C.

5. The particles of claim 1, wherein when the particles have a mean micrometric size, the particles have a coefficient of uniformity of less than or equal to 0.45.

6. The particles of claim 1, wherein when the particles have a mean submicrometric size, the particles have a polydispersity index of less than or equal to 0.35.

7. The particles of claim 1, wherein the particles are substantially spherical.

8. The particles of claim 1, wherein the oily phase comprises at least one non-volatile oil.

9. The particles of claim 1, wherein the oily phase is present in an amount of from 30% to 99% by weight relative to a total weight of the particles.

10. The particles of claim 1, wherein the oily phase comprises at least one oil selected from the group consisting of a plant oil, an animal oil, a synthetic oil and a mineral oil.

11. The particles of claim 1, wherein the oily phase comprises at least one oil selected from the group consisting of squalane, isononyl isononanoate, isopropyl lauroylsarcosinate and octyldodecanol.

12. The particles of claim 1, wherein the gelling polymer comprises at least one polymer selected from the group consisting of a semi-crystalline polymer, a polyamide, a silicone polyamide, a polysaccharide monoalkyl ester, a polysaccharide polyalkyl ester, and a diblock and/or triblock and/or multiblock and/or radial-block copolymer.

13. The particles of claim 1, wherein the gelling polymer comprises at least one polymer selected from the group consisting of ethylenediamine/stearyl dimerdilinoleate and a semi-crystalline polymer of poly-C₁₀₋₃₀ alkyl acrylate type.

14. The particles of claim 1, wherein the gelling polymer is present in an amount of from 1% to 60% by weight relative to a total weight of the particles.

15. The particles of claim 1, wherein a weight ratio of the gelling polymer to the oily phase of the particles is from 0.1 to 1.

16. The particles of claim 1, further comprising at least one active substance.

17. The particles of claim 16, wherein the active substance comprises at least one member selected from the group consisting of an antibacterial agent, an antifungal agent, a sebo-regulatory agent, a sebo-stimulatory agent, a keratolytic agent, an agent for treating acne, an antibiotic of macrolide structure, an agent for inhibiting hair loss, an agent for inhibiting hair growth, an antidandruff agent, an antioxidant, an astringent, a pore-reducing agent, an antiperspirant, a vitamin and an anti-inflammatory agent.

18. The particles of claim 16, wherein the active substance is present in an amount of from 0.5% to 40% by weight relative to a total weight of the particles.

19. An aqueous and/or water-soluble phase dispersion comprising the particles of claim 1.

20. The dispersion of claim 19, further comprising at least one surfactant selected from the group consisting of an ionic surfactant and a nonionic surfactant.

21. The dispersion of claim 19, further comprising at least one hydrophobic emulsifying polymer.

22. The dispersion of claim 19, further comprising at least one hydrophilic gelling polymer.

23. The dispersion of claim 19, wherein the particles are dispersed in an aqueous phase, and the particles represent an oily volume fraction of from 60% to 80% by weight relative to a total weight of the dispersion.

24. A cosmetic or dermatological composition comprising the particles of claim 1.

25. A method of treating greasy skin comprising applying a composition comprising the particles of claim 1 to the skin.

26. A method of preparing a dermatological composition for treating acne comprising employing the particles of claim 1.

27. A non-therapeutic process for making up and/or caring for the skin, comprising applying a composition comprising the particles of claim 1 to the skin.

28. A process for manufacturing a dispersion of calibrated and spherical oily particles comprising at least one oily phase structured with at least one gelling polymer and having a mean size of less than or equal to 15 μm, the process comprising:
emulsifying a mixture of at least one oil or an oily phase and at least one oily-phase-gelling polymer with an aqueous and/or water-soluble phase at a temperature above a gel point of the polymer;
subjecting the mixture to a process leading to production of the oily particles at a temperature at least 5° C. above a melting point of the mixture; and
cooling the obtained particle dispersion.

29. The process of claim 28, wherein the gelling polymer comprises at least one polymer selected from the group consisting of a semi-crystalline polymer, a polyamide, a silicone polyamide, a polysaccharide monoalkyl ester, a polysaccharide polyalkyl ester, and a diblock and/or triblock and/or multiblock and/or radial-block copolymer.

30. The process of claim 28, wherein the gelling polymer comprises at least one polymer selected from the group consisting of ethylenediamine/stearyl dimerdilinoleate and a semi-crystalline polymer of poly-C₁₀₋₃₀ alkyl acrylate type.

31. The process of claim 28, wherein the gelling polymer is present in an amount of from 1% to 80% by weight relative to a total weight of the particles.

32. The process of claim 28, wherein the particles obtained by the process have a mean size of from about 1 to about 15 micrometers.

33. The process of claim 28, wherein the process employs at least one technique selected from the group consisting of controlled shear of viscoelastic emulsions, a process using a colloidal mill, a process using a static mixer, a process using a micromixer, a process using a frame paddle, a process using an extruder-blender, a process using a porous membrane, a process using maturation control, a process using swelling of a “templating agent” latex, a process using Rayleigh instabilities, and a process using fractionation of a polydisperse emulsion.

34. The process of claim 28, wherein the particles obtained by the process have a mean size of from about 150 nm to about 1 μm.

35. The process of claim 28, wherein the process employs at least one technique selected from the group consisting of a process developing a shear and a process involving a phase inversion.

36. The process of claim 28, wherein the temperature above the gel point of the polymer is greater than 40° C.

37. The process of claim 28, wherein the mixture further comprises an active substance.

38. Calibrated and spherical oily particles obtained by the process of claim 28.

39. Calibrated and spherical oily particles, comprising at least one oily phase structured with at least one gelling polymer, wherein:
the particles have a mean size of less than or equal to 15 μm;
the gelling polymer comprises at least one member selected from the group consisting of a semi-crystalline polymer, a polyamide, a silicone polyamide, a polysaccharide monoalkyl ester or polyalkyl ester, with the exception of fatty acid esters of dextrin, and a diblock and/or triblock and/or multiblock and/or radial-block copolymer; and
the particles have a circularity index of from 0.9 to 1.

40. The particles of claim 39, wherein the particles have a mean size of from 150 nm to 11 μm.

41. The particles of claim 39, wherein the structured oily phase has a melting point of from 50° C. to 90° C.

42. The particles of claim 39, wherein when the particles have a mean micrometric size, the particles have a coefficient of uniformity of less than or equal to 0.45.

43. The particles of claim 39, wherein when the particles have a mean submicrometric size, the particles have a polydispersity index of less than or equal to 0.35.

44. The particles of claim 39, wherein the oily phase comprises at least one non-volatile oil.

45. The particles of claim 39, wherein the oily phase is present in an amount of from 30% to 99% by weight relative to a total weight of the particles.

46. The particles of claim 39, wherein the oily phase comprises at least one oil selected from the group consisting of a plant oil, an animal oil, a synthetic oil and a mineral oil.

47. The particles of claim 39, wherein the oily phase comprises at least one oil selected from the group consisting of squalane, isononyl isononanoate, isopropyl lauroylsarcosinate and octyldodecanol.

48. The particles of claim 39, wherein the gelling polymer comprises at least one polymer selected from the group consisting of ethylenediamine/stearyl dimerdilinoleate and a semi-crystalline polymer of poly-C₁₀₋₃₀ alkyl acrylate type.

49. The particles of claim 39, wherein the gelling polymer is present in an amount of from 1% to 60% by weight relative to a total weight of the particles.

50. The particles of claim 39, wherein a weight ratio of the gelling polymer to the oily phase of the particles is from 0.1 to 1.

51. The particles of claim 39, further comprising at least one active substance.

52. An aqueous and/or water-soluble phase dispersion comprising the particles of claim 39.

53. A cosmetic or dermatological composition comprising the particles of claim 39.

54. A method of treating greasy skin comprising applying a composition comprising the particles of claim 39 to the skin.

55. A method of preparing a dermatological composition for treating acne comprising employing the particles of claim 39.

56. A non-therapeutic process for making up and/or caring for the skin, comprising applying a composition comprising the particles of claim 39 to the skin.

57. Calibrated and spherical oily particles, obtained from an oily phase structured with at least one gelling polymer, wherein:
the gelling polymer is of a nature and/or in present in an amount sufficient to give the oily phase a viscosity of greater than or equal to 750 Pa·s at a shear of 1 s⁻¹ at 25° C.; and
the particles have a circularity index of from 0.9 to 1.

58. The particles of claim 57, wherein the particles have a mean size of from 150 nm to 11 μm.

59. The particles of claim 57, wherein the structured oily phase has a melting point of from 50° C. to 90° C.

60. The particles of claim 57, wherein when the particles have a mean micrometric size, the particles have a coefficient of uniformity of less than or equal to 0.45.

61. The particles of claim 57, wherein when the particles have a mean submicrometric size, the particles have a polydispersity index of less than or equal to 0.35.

62. The particles of claim 57, wherein the oily phase comprises at least one non-volatile oil.

63. The particles of claim 57, wherein the oily phase is present in an amount of from 30% to 99% by weight relative to a total weight of the particles.

64. The particles of claim 57, wherein the oily phase comprises at least one oil selected from the group consisting of a plant oil, an animal oil, a synthetic oil and a mineral oil.

65. The particles of claim 57, wherein the oily phase comprises at least one oil selected from the group consisting of squalane, isononyl isononanoate, isopropyl lauroylsarcosinate and octyldodecanol.

66. The particles of claim 57, wherein the gelling polymer comprises at least one polymer selected from the group consisting of a semi-crystalline polymer, a polyamide, a silicone polyamide, a polysaccharide monoalkyl ester, a polysaccharide polyalkyl ester, and a diblock and/or triblock and/or multiblock and/or radial-block copolymer.

67. The particles of claim 57, wherein the gelling polymer comprises at least one polymer selected from the group consisting of ethylenediamine/stearyl dimerdilinoleate and a semi-crystalline polymer of poly-C₁₀₋₃₀ alkyl acrylate type.

68. The particles of claim 57, wherein the gelling polymer is present in an amount of from 1% to 60% by weight relative to a total weight of the particles.

69. The particles of claim 57, wherein a weight ratio of the gelling polymer to the oily phase of the particles is from 0.1 to 1.

70. The particles of claim 57, further comprising at least one active substance.

71. An aqueous and/or water-soluble phase dispersion comprising the particles of claim 57.

72. A cosmetic or dermatological composition comprising the particles of claim 57.

73. A method of treating greasy skin comprising applying a composition comprising the particles of claim 57 to the skin.

74. A method of preparing a dermatological composition for treating acne comprising employing the particles of claim 57.

75. A non-therapeutic process for making up and/or caring for the skin, comprising applying a composition comprising the particles of claim 57 to the skin.
